SEQUENCE LISTING (21 sequences)

SEQ ID NO: 1 - DNA, 888 bp, synthetic construct
  misc_feature 1..888: note = Pseudomonas aeruginosa RhlA nucleotide sequence
  source 1..888: mol_type = other DNA
atgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgcgggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgcagcgcctgaaagccagcaaccatcagcacatggcttcgctggccaccggcgaatacgagcaggcgcgctttcacatcgaccaggtgctggcgctcaacgatcggggctacttggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggcagcggtacgcgtgcaccgcgccctgctcgagcacctgctgaagcaaccggagccgcagcgggcggaacgcgcggcgggattccacgagatggccatcggctacgcctga

SEQ ID NO: 2 - DNA, 1281 bp, synthetic construct
  misc_feature 1..1281: note = Pseudomonas aeruginosa RhlB nucleotide
  source 1..1281: mol_type = other DNA
atgcacgccatcctcatcgccatcggctcggccggcgacgtatttcccttcatcggcctggcccggaccctgaaactgcgcgggcaccgcgtgagcctctgcaccatcccggtgtttcgcgacgcggtggagcagcacggcatcgcgttcgtcccgctgagcgacgaactgacctaccgccggaccatgggcgatccgcgcctgtgggaccccaagacgtccttcggcgtgctctggcaagccatcgccgggatgatcgagccggtctacgagtacgtctcggcgcagcgccatgacgacatcgtggtggtcggctcgctatgggcgctgggcgcacgcatcgctcacgagaagtacgggattccctacctgtccgcgcaggtctcgccatcgaccctgttgtcggcgcacctgccgccggtacaccccaagttcaacgtgcccgagcagatgccgctggcgatgcgcaagctgctctggcgctgcatcgagcgcttcaagctggatcgcacctgcgcgccggagatcaacgcggtgcgccgcaaggtcggcctggaaacgccggtgaagcgcatcttcacccaatggatgcattcgccgcagggcgtggtctgcctgttcccggcctggttcgcgccgccccagcaggattggccgcaacccctgcacatgaccggcttcccgctgttcgacggcagtatcccggggaccccgctcgacgacgaactgcaacgctttctcgatcagggcagccggccgctggtgttcacccagggctcgaccgaacacctgcagggcgacttctacgccatggccctgcgcgcgctggaacgcctcggcgcgcgtgggatcttcctcaccggcgccggccaggaaccgctgcgcggcttgccgaaccacgtgctgcagcgcgcctacgcgccactgggagccttgctgccatcgtgcgccgggctggtccatccgggcggtatcggcgccatgagcctagccttggcggcgggggtgccgcaggtgctgctgccctgtgcccacgaccagttcgacaatgccgaacggctggtccggctcggctgcgggatgcgcctgggcgtgccgttgcgcgagcaggagttgcgcggggcgctgtggcgcttgctcgaggacccggccatggcggcggcctgtcggcgtttcatggaattgtcacaaccgcacagtatcgcttgcggtaaagcggcccaggtggtcgaacgttgtcatagggagggggatgctcgatggctgaaggctgcgtcctga

SEQ ID NO: 3 - DNA, 978 bp, synthetic construct
  misc_feature 1..978: note = Pseudomonas aeruginosa RhlC nucleotide
  source 1..978: mol_type = other DNA
atggaccggatagacatgggcgtgctggtggtactgttcaatcctggcgacgacgacctggaacaccttggcgaactggcggcggcgtttccgcaactgcgcttccttgccgtcgacaactcaccgcacagcgatccgcagcgcaatgcccggctgcgcgggcaaggcatcgccgtgctgcaccacggcaaccggcagggcatcgccggcgccttcaaccagggactcgacgcgctattccggcgtggcgtgcagggtgtgctgctgctcgaccaggactcccgtcccggcggcgccttcctcgccgcccagtggcgcaacctgcaggcgcgcaacggtcaggcctgcctgctcggcccacggatcttcgaccggggtgaccggcgcttcctgccggccatccatctcgacggactgacgctcaggcaattgtctctggacggcctgacgaccccgcagcgcacctcgttcctgatctcctccggctgcctgctgacccgcgaggcctaccagcgcctcggccacttcgacgaggaactgttcatcgaccacgtggacaccgaatacagcctgcgcgcccaggcgctggacgtgcccctgtacgtcgacccgcggctggtcctcgagcaccgcatcggcacgcgcaagacccgccgcctcggcggtctcagcctcagcgcgatgaaccacgccccgctgcgccgctactacctggcgcgcaacggcctgctggtcctgcgccgctacgcccggtcctcgccgctggccctgctggcgaacctgccgaccctgacccagggcctcgcggtgctcctgctcgaacgcgacaagctgctcaagctgcgctgcctgggctggggcctgtgggacggcctgcggggacgcggcggcgcgctggagaccaaccgcccgcgcctgctgaagcgcctcgccggcccggccgtggcgtccgtagcttccggcaaggccaaggcctag

SEQ ID NO: 4 - DNA, 570 bp, synthetic construct
  misc_feature 1..570: note = RhlM nucleotide sequence
  source 1..570: mol_type = other DNA
atgaacatcgcccagagcatcgccatcgtggtgccgtggaccctgtggctgctgtactggatcgccaccagcaaccgcgtgaaggagaccgcccgcaaggaggccagcctgagccgcaccctgcagagcatcccgctgatcggcggcggcgccctgatcatcctgccggacttcaacaccgccgccctgagcttcgacccgcacgccatcggcccgctgcagctggtgggcctggccgtgctgctggccggcctggccttcagcgtgtgggcccgcctgcacctgggcaccaactggagcgtgagcgtgaccctgaaggaggaccacgagctggtgcgcagcggcccgtacggcctggtgcgccacccgatctacaccggctgcctgctggccctgctgggcgccgtgctgatcggcgcccagtggcgcggcgtggccggcctggtgctgatcttcgccagcctggcctacaaggtgcgcgtggaggagagctggctgaccggccacttcggccgcgcctacagccagtaccgccgcgacgtggccgccctgatcccgggcctgttctga

SEQ ID NO: 5 - DNA, 1230 bp, synthetic construct
  misc_feature 1..1230: note = Hydrolase nucleotide sequence
  source 1..1230: mol_type = other DNA
atggagttcccgatggcccagagcaacatcatcgccggcatggacctgaaccgcctggaccgcatcgccgagcacctggaccgcgcctacctgcacccgggcaagctggccggcaccatgaccctggtggcccgccgcggcgaggtggtgtactgccaggcccagggcctgcgcgacgtggagcgccagctgccggtggagcgcgacaccctgttccgcatctacagcatgaccaagccgatcaccagcatcgccctgatgcagctgtacgagcagggccgcttcctgctggacgagccggtgcacaagtacatcccgacctggaagaacctgcgcgtgtacaagaccggcagccacccgcagatgctgaccaccgccccgcagcgcccgatgaccatccgcgacctgctgacccaccagagcggcctgacctacggcttcatgaaccgcaccaacgtggacgccgcctaccgcagcctgaagctggacggcggcccgggccacaccctggaccgcctgatcgacgagctggcccgcctgccgctggagttcagcccgggcaccgcctggaactacagcgtggccaccgacgtgtgcggctacctggtgcagctgctgagcggcatgagcctggacgactacttcagcaagcacatcttccagccgctgggcatgccggacaccttcttcaccgtgccggccgagaagctgagccgcttcgccgcctgctacgagtaccagccgggcgacagcttcagcctgcaggacgacccgcagggcagcgccttcgccaaggcccacggctacctgagcggcggcggcggcctggtgagctgcgtggacgactactaccgcttcgcccaggccctggccaacggcggcgagctggacggcgcccgcatcatcggccgcaagaccctggagttcatgcgcatgaaccacctgccggacaacaagggcctgccggacgtggccatcggcagcttcagcgagaccccgtacgacggcaccggcttcggcctgggcttcagcgtgaagctggacgtggccaagagccagaccgtgggcagcgtgggcgagtacggctggggcggcatggccagcaccaacttcttcatcgacccggaggaggacctgctgatggtgttcatgacccagctgatcccgagcagcacctacgccgtgcgccaggagctgcgcgccatcatcaacggcgccctggtggactga

SEQ ID NO: 6 - DNA, 1301 bp, synthetic construct
  misc_feature 1..1301: note = Hydrolase nucleotide sequence with N-His/thrombin
  source 1..1301: mol_type = other DNA
ccatgggcagcagccatcatcatcatcatcacagcagcggcctggtgccgcgcggcagccatatggagttcccgatggcgcagagcaacatcattgcgggtatggacctgaaccgtctggatcgtatcgcggaacatctggaccgtgcgtacctgcacccgggtaaactggcgggtaccatgaccctggttgcgcgtcgtggcgaggtggtttattgccaggcgcaaggtctgcgtgacgtggagcgtcaactgccggttgaacgtgataccctgttccgtatttacagcatgaccaagccgatcaccagcattgcgctgatgcagctgtatgagcaaggccgttttctgctggacgaaccggtgcacaagtacatcccgacctggaaaaacctgcgtgtttataagaccggtagccacccgcagatgctgaccaccgcgccgcaacgtccgatgaccattcgtgatctgctgacccaccagagcggcctgacctacggtttcatgaaccgtaccaacgtggacgcggcgtatcgtagcctgaaactggatggtggcccgggtcacaccctggaccgtctgatcgatgagctggcgcgtctgccgctggaatttagcccgggtaccgcgtggaactacagcgtggcgaccgacgtttgcggctatctggttcagctgctgagcggtatgagcctggacgattacttcagcaaacacatttttcaaccgctgggcatgccggacaccttctttaccgttccggcggagaagctgagccgttttgcggcgtgctacgaatatcagccgggtgatagcttcagcctgcaggacgatccgcaaggcagcgcgtttgcgaaggcgcacggttatctgagcggtggcggtggcctggttagctgcgttgacgattactatcgttttgcgcaggcgctggcgaacggtggcgagctggatggtgcgcgtatcattggtcgtaaaaccctggaattcatgcgtatgaaccacctgccggacaacaagggcctgccggatgttgcgatcggtagctttagcgagaccccgtacgacggtaccggcttcggtctgggctttagcgtgaagctggatgttgcgaaaagccagaccgtgggtagcgttggcgaatatggttggggtggcatggcgagcaccaacttctttatcgacccggaggaagatctgctgatggtgttcatgacccagctgattccgagcagcacctacgcggttcgtcaagaactgcgtgcgatcattaacggtgcgctggttgactaataactcgag

SEQ ID NO: 7 - protein, 295 aa, synthetic construct
  REGION 1..295: note = RhlA protein sequence
MRRESLLVSVCKGLRVHVERVGQDPGRSTVMLVNGAMATTASFARTCKCLAEHFNVVLFDLPFAGQSRQHNPQRGLITKDDEVEILLALIERFEVNHLVSASWGGISTLLALSRNPRGIRSSVVMAFAPGLNQAMLDYVGRAQALIELDDKSAIGHLLNETVGKYLPQRLKASNHQHMASLATGEYEQARFHIDQVLALNDRGYLACLERIQSHVHFINGSWDEYTTAEDARQFRDYLPHCSFSRVEGTGHFLDLESKLAAVRVHRALLEHLLKQPEPQRAERAAGFHEMAIGYA

SEQ ID NO: 8 - protein, 426 aa, synthetic construct
  REGION 1..426: note = RhlB protein sequence
MHAILIAIGSAGDVFPFIGLARTLKLRGHRVSLCTIPVFRDAVEQHGIAFVPLSDELTYRRTMGDPRLWDPKTSFGVLWQAIAGMIEPVYEYVSAQRHDDIVVVGSLWALGARIAHEKYGIPYLSAQVSPSTLLSAHLPPVHPKFNVPEQMPLAMRKLLWRCIERFKLDRTCAPEINAVRRKVGLETPVKRIFTQWMHSPQGVVCLFPAWFAPPQQDWPQPLHMTGFPLFDGSIPGTPLDDELQRFLDQGSRPLVFTQGSTEHLQGDFYAMALRALERLGARGIFLTGAGQEPLRGLPNHVLQRAYAPLGALLPSCAGLVHPGGIGAMSLALAAGVPQVLLPCAHDQFDNAERLVRLGCGMRLGVPLREQELRGALWRLLEDPAMAAACRRFMELSQPHSIACGKAAQVVERCHREGDARWLKAAS

SEQ ID NO: 9 - protein, 325 aa, synthetic construct
  REGION 1..325: note = RhlC protein sequence
MDRIDMGVLVVLFNPGDDDLEHLGELAAAFPQLRFLAVDNSPHSDPQRNARLRGQGIAVLHHGNRQGIAGAFNQGLDALFRRGVQGVLLLDQDSRPGGAFLAAQWRNLQARNGQACLLGPRIFDRGDRRFLPAIHLDGLTLRQLSLDGLTTPQRTSFLISSGCLLTREAYQRLGHFDEELFIDHVDTEYSLRAQALDVPLYVDPRLVLEHRIGTRKTRRLGGLSLSAMNHAPLRRYYLARNGLLVLRRYARSSPLALLANLPTLTQGLAVLLLERDKLLKLRCLGWGLWDGLRGRGGALETNRPRLLKRLAGPAVASVASGKAKA

SEQ ID NO: 10 - protein, 189 aa, synthetic construct
  REGION 1..189: note = RhlM protein sequence
MNIAQSIAIVVPWTLWLLYWIATSNRVKETARKEASLSRTLQSIPLIGGGALIILPDFNTAALSFDPHAIGPLQLVGLAVLLAGLAFSVWARLHLGTNWSVSVTLKEDHELVRSGPYGLVRHPIYTGCLLALLGAVLIGAQWRGVAGLVLIFASLAYKVRVEESWLTGHFGRAYSQYRRDVAALIPGLF

SEQ ID NO: 11 - protein, 409 aa, synthetic construct
  REGION 1..409: note = Hydrolase protein sequence
MEFPMAQSNIIAGMDLNRLDRIAEHLDRAYLHPGKLAGTMTLVARRGEVVYCQAQGLRDVERQLPVERDTLFRIYSMTKPITSIALMQLYEQGRFLLDEPVHKYIPTWKNLRVYKTGSHPQMLTTAPQRPMTIRDLLTHQSGLTYGFMNRTNVDAAYRSLKLDGGPGHTLDRLIDELARLPLEFSPGTAWNYSVATDVCGYLVQLLSGMSLDDYFSKHIFQPLGMPDTFFTVPAEKLSRFAACYEYQPGDSFSLQDDPQGSAFAKAHGYLSGGGGLVSCVDDYYRFAQALANGGELDGARIIGRKTLEFMRMNHLPDNKGLPDVAIGSFSETPYDGTGFGLGFSVKLDVAKSQTVGSVGEYGWGGMASTNFFIDPEEDLLMVFMTQLIPSSTYAVRQELRAIINGALVD

SEQ ID NO: 12 - protein, 429 aa, synthetic construct
  REGION 1..429: note = Hydrolase protein sequence with N-histidine/thrombin
MGSSHHHHHHSSGLVPRGSHMEFPMAQSNIIAGMDLNRLDRIAEHLDRAYLHPGKLAGTMTLVARRGEVVYCQAQGLRDVERQLPVERDTLFRIYSMTKPITSIALMQLYEQGRFLLDEPVHKYIPTWKNLRVYKTGSHPQMLTTAPQRPMTIRDLLTHQSGLTYGFMNRTNVDAAYRSLKLDGGPGHTLDRLIDELARLPLEFSPGTAWNYSVATDVCGYLVQLLSGMSLDDYFSKHIFQPLGMPDTFFTVPAEKLSRFAACYEYQPGDSFSLQDDPQGSAFAKAHGYLSGGGGLVSCVDDYYRFAQALANGGELDGARIIGRKTLEFMRMNHLPDNKGLPDVAIGSFSETPYDGTGFGLGFSVKLDVAKSQTVGSVGEYGWGGMASTNFFIDPEEDLLMVFMTQLIPSSTYAVRQELRAIINGALVD

SEQ ID NO: 13 - DNA, 1230 bp, synthetic construct
  misc_feature 1..1230: note = Hydrolase Est-14 nucleotide sequence
  source 1..1230: mol_type = other DNA
atggaatttccaatggcgcagagcaatattatcgcgggtatggacctgaaccgactggatcgtatcgccgagcacctggatcgggcgtaccttcatccaggcaagctagcaggcaccatgaccctggtcgcccgccgtggcgaggtagtctactgccaggctcaggggctgcgtgatgtggagcgccagttaccagtagaacgcgatacgctgtttcgtatctattcgatgaccaagccgattacctctattgccttgatgcagctctacgaacagggccgttttctactggatgagccagttcacaagtacattccgacctggaaaaacctgcgcgtatataaaaccggcagtcatccgcaaatgctgactacggcaccgcagcgtccgatgaccattcgtgacctgttaacccaccagtccggccttacctacggcttcatgaatcgtaccaatgtggacgcggcctaccgcagcctcaagctggatggcggcccggggcacacgctcgatcgcttgatcgacgagctggctcgattgcctctggagttttcacccggcacggcctggaattactccgtcgcaaccgacgtgtgtggctatctggtgcaattgctctccggtatgagtctggacgattatttcagcaagcacatttttcagccgctgggcatgccagacaccttctttaccgtgccagcagaaaaactgagtcgctttgccgcctgttatgagtatcaaccgggtgacagcttcagcctgcaggatgatccgcaaggctcagcctttgccaaggcgcatggctatctgtctggcggcggcggcctggtctcgtgtgtcgacgactactaccgctttgctcaggccctggccaacggcggcgaactcgatggcgcgcgtattatcggccgcaagactctggaattcatgcgcatgaaccatctccccgataacaagggcttgcccgacgtcgccattggctcctttagcgagactccctacgacggcaccggttttggcttgggtttttcggtcaagctcgacgttgccaaatcccagaccgtcggttcagtgggcgaatacggttggggtggcatggcgagcaccaacttcttcatcgatccagaagaagacctgttgatggtgtttatgacgcagctgattccttcttctacctatgcggtgcggcaggagttacgggcgatcattaatggggcgttggtggattga

SEQ ID NO: 14 - protein, 409 aa, synthetic construct; identical to SEQ ID NO: 11
  REGION 1..409: note = Hydrolase Est-14 protein sequence

SEQ ID NO: 15 - DNA, 3826 bp, synthetic construct
  misc_feature 1..3826: note = pPG2 insert
  source 1..3826: mol_type = other DNA
ggtacccttggactcctgttgatagatccagtaatgacctcagaactccatctggatttgttcagaacgctcggttgccgccgggcgttttttattggtgagaatccagagctcttgacaaggtcggaaaattgaagtataatatcagtagggggttctagagatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgcgggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgcagcgcctgaaagccagcaaccatcagcacatggcttcgctggccaccggcgaatacgagcaggcgcgctttcacatcgaccaggtgctggcgctcaacgatcggggctacttggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggcagcggtacgcgtgcaccgcgccctgctcgagcacctgctgaagcaaccggagccgcagcgggcggaacgcgcggcgggattccacgagatggccatcggctacgcctgaacccttgacctgcgaagacccggcctggccgggctttgcggttgcataacgcacggagtagccccatgcacgccatcctcatcgccatcggctcggccggcgacgtatttcccttcatcggcctggcccggaccctgaaactgcgcgggcaccgcgtgagcctctgcaccatcccggtgtttcgcgacgcggtggagcagcacggcatcgcgttcgtcccgctgagcgacgaactgacctaccgccggaccatgggcgatccgcgcctgtgggaccccaagacgtccttcggcgtgctctggcaagccatcgccgggatgatcgagccggtctacgagtacgtctcggcgcagcgccatgacgacatcgtggtggtcggctcgctatgggcgctgggcgcacgcatcgctcacgagaagtacgggattccctacctgtccgcgcaggtctcgccatcgaccctgttgtcggcgcacctgccgccggtacaccccaagttcaacgtgcccgagcagatgccgctggcgatgcgcaagctgctctggcgctgcatcgagcgcttcaagctggatcgcacctgcgcgccggagatcaacgcggtgcgccgcaaggtcggcctggaaacgccggtgaagcgcatcttcacccaatggatgcattcgccgcagggcgtggtctgcctgttcccggcctggttcgcgccgccccagcaggattggccgcaacccctgcacatgaccggcttcccgctgttcgacggcagtatcccggggaccccgctcgacgacgaactgcaacgctttctcgatcagggcagccggccgctggtgttcacccagggctcgaccgaacacctgcagggcgacttctacgccatggccctgcgcgcgctggaacgcctcggcgcgcgtgggatcttcctcaccggcgccggccaggaaccgctgcgcggcttgccgaaccacgtgctgcagcgcgcctacgcgccactgggagccttgctgccatcgtgcgccgggctggtccatccgggcggtatcggcgccatgagcctagccttggcggcgggggtgccgcaggtgctgctgccctgtgcccacgaccagttcgacaatgccgaacggctggtccggctcggctgcgggatgcgcctgggcgtgccgttgcgcgagcaggagttgcgcggggcgctgtggcgcttgctcgaggacccggccatggcggcggcctgtcggcgtttcatggaattgtcacaaccgcacagtatcgcttgcggtaaagcggcccaggtggtcgaacgttgtcatagggagggggatgctcgatggctgaaggctgcgtcctgaacggtgctggcataacagatagggttgccatgattttgccgtatcggcaaggctgcgcgcttgacagcgtcataccccgggccaattctgctgtgatgcattttatcgatcagggcttactgcaatggagttcccgatggcccagagcaacatcatcgccggcatggacctgaaccgcctggaccgcatcgccgagcacctggaccgcgcctacctgcacccgggcaagctggccggcaccatgaccctggtggcccgccgcggcgaggtggtgtactgccaggcccagggcctgcgcgacgtggagcgccagctgccggtggagcgcgacaccctgttccgcatctacagcatgaccaagccgatcaccagcatcgccctgatgcagctgtacgagcagggccgcttcctgctggacgagccggtgcacaagtacatcccgacctggaagaacctgcgcgtgtacaagaccggcagccacccgcagatgctgaccaccgccccgcagcgcccgatgaccatccgcgacctgctgacccaccagagcggcctgacctacggcttcatgaaccgcaccaacgtggacgccgcctaccgcagcctgaagctggacggcggcccgggccacaccctggaccgcctgatcgacgagctggcccgcctgccgctggagttcagcccgggcaccgcctggaactacagcgtggccaccgacgtgtgcggctacctggtgcagctgctgagcggcatgagcctggacgactacttcagcaagcacatcttccagccgctgggcatgccggacaccttcttcaccgtgccggccgagaagctgagccgcttcgccgcctgctacgagtaccagccgggcgacagcttcagcctgcaggacgacccgcagggcagcgccttcgccaaggcccacggctacctgagcggcggcggcggcctggtgagctgcgtggacgactactaccgcttcgcccaggccctggccaacggcggcgagctggacggcgcccgcatcatcggccgcaagaccctggagttcatgcgcatgaaccacctgccggacaacaagggcctgccggacgtggccatcggcagcttcagcgagaccccgtacgacggcaccggcttcggcctgggcttcagcgtgaagctggacgtggccaagagccagaccgtgggcagcgtgggcgagtacggctggggcggcatggccagcaccaacttcttcatcgacccggaggaggacctgctgatggtgttcatgacccagctgatcccgagcagcacctacgccgtgcgccaggagctgcgcgccatcatcaacggcgccctggtggactgaagcgcagggcgcgccggtcggcgcgataaaacgaaaggctcagtcgaaagactgggcctttcgttttataagctt

SEQ ID NO: 16 - DNA, 4654 bp, synthetic construct
  misc_feature 1..4654: note = pPG6 insert
  source 1..4654: mol_type = other DNA
ggtacccttggactcctgttgatagatccagtaatgacctcagaactccatctggatttgttcagaacgctcggttgccgccgggcgttttttattggtgagaatccagagctcttgacaaggtcggaaaattgaagtataatatcagtagggggttctagagatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgcgggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgcagcgcctgaaagccagcaaccatcagcacatggcttcgctggccaccggcgaatacgagcaggcgcgctttcacatcgaccaggtgctggcgctcaacgatcggggctacttggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggcagcggtacgcgtgcaccgcgccctgctcgagcacctgctgaagcaaccggagccgcagcgggcggaacgcgcggcgggattccacgagatggccatcggctacgcctgaacccttgacctgcgaagacccggcctggccgggctttgcggttgcataacgcacggagtagccccatgcacgccatcctcatcgccatcggctcggccggcgacgtatttcccttcatcggcctggcccggaccctgaaactgcgcgggcaccgcgtgagcctctgcaccatcccggtgtttcgcgacgcggtggagcagcacggcatcgcgttcgtcccgctgagcgacgaactgacctaccgccggaccatgggcgatccgcgcctgtgggaccccaagacgtccttcggcgtgctctggcaagccatcgccgggatgatcgagccggtctacgagtacgtctcggcgcagcgccatgacgacatcgtggtggtcggctcgctatgggcgctgggcgcacgcatcgctcacgagaagtacgggattccctacctgtccgcgcaggtctcgccatcgaccctgttgtcggcgcacctgccgccggtacaccccaagttcaacgtgcccgagcagatgccgctggcgatgcgcaagctgctctggcgctgcatcgagcgcttcaagctggatcgcacctgcgcgccggagatcaacgcggtgcgccgcaaggtcggcctggaaacgccggtgaagcgcatcttcacccaatggatgcattcgccgcagggcgtggtctgcctgttcccggcctggttcgcgccgccccagcaggattggccgcaacccctgcacatgaccggcttcccgctgttcgacggcagtatcccggggaccccgctcgacgacgaactgcaacgctttctcgatcagggcagccggccgctggtgttcacccagggctcgaccgaacacctgcagggcgacttctacgccatggccctgcgcgcgctggaacgcctcggcgcgcgtgggatcttcctcaccggcgccggccaggaaccgctgcgcggcttgccgaaccacgtgctgcagcgcgcctacgcgccactgggagccttgctgccatcgtgcgccgggctggtccatccgggcggtatcggcgccatgagcctagccttggcggcgggggtgccgcaggtgctgctgccctgtgcccacgaccagttcgacaatgccgaacggctggtccggctcggctgcgggatgcgcctgggcgtgccgttgcgcgagcaggagttgcgcggggcgctgtggcgcttgctcgaggacccggccatggcggcggcctgtcggcgtttcatggaattgtcacaaccgcacagtatcgcttgcggtaaagcggcccaggtggtcgaacgttgtcatagggagggggatgctcgatggctgaaggctgcgtcctgaacggtgctggcataacagatagggttgccatgattttgccgtatcggcaaggctgcgcgcttgacagcgtcataccccgggccaattctgctgtgatgcattttatcgatcagggcttactgcaatggagttcccgatggcccagagcaacatcatcgccggcatggacctgaaccgcctggaccgcatcgccgagcacctggaccgcgcctacctgcacccgggcaagctggccggcaccatgaccctggtggcccgccgcggcgaggtggtgtactgccaggcccagggcctgcgcgacgtggagcgccagctgccggtggagcgcgacaccctgttccgcatctacagcatgaccaagccgatcaccagcatcgccctgatgcagctgtacgagcagggccgcttcctgctggacgagccggtgcacaagtacatcccgacctggaagaacctgcgcgtgtacaagaccggcagccacccgcagatgctgaccaccgccccgcagcgcccgatgaccatccgcgacctgctgacccaccagagcggcctgacctacggcttcatgaaccgcaccaacgtggacgccgcctaccgcagcctgaagctggacggcggcccgggccacaccctggaccgcctgatcgacgagctggcccgcctgccgctggagttcagcccgggcaccgcctggaactacagcgtggccaccgacgtgtgcggctacctggtgcagctgctgagcggcatgagcctggacgactacttcagcaagcacatcttccagccgctgggcatgccggacaccttcttcaccgtgccggccgagaagctgagccgcttcgccgcctgctacgagtaccagccgggcgacagcttcagcctgcaggacgacccgcagggcagcgccttcgccaaggcccacggctacctgagcggcggcggcggcctggtgagctgcgtggacgactactaccgcttcgcccaggccctggccaacggcggcgagctggacggcgcccgcatcatcggccgcaagaccctggagttcatgcgcatgaaccacctgccggacaacaagggcctgccggacgtggccatcggcagcttcagcgagaccccgtacgacggcaccggcttcggcctgggcttcagcgtgaagctggacgtggccaagagccagaccgtgggcagcgtgggcgagtacggctggggcggcatggccagcaccaacttcttcatcgacccggaggaggacctgctgatggtgttcatgacccagctgatcccgagcagcacctacgccgtgcgccaggagctgcgcgccatcatcaacggcgccctggtggactgaagcgcagggcgcgccggtcggcgcgataaaacgaaaggctcagtcgaaagactgggcctttcgttttatcgggccccccctcgaggtcgacggtatcgatacttggactcctgttgatagatccagtaatgacctcagaactccatctggatttgttcagaacgctcggttgccgccgggcgttttttattggtgagaatccagagctcttgacaaggtcggaaaattgaagtataatatcagtagggggttctagagatgaacatcgcccagagcatcgccatcgtggtgccgtggaccctgtggctgctgtactggatcgccaccagcaaccgcgtgaaggagaccgcccgcaaggaggccagcctgagccgcaccctgcagagcatcccgctgatcggcggcggcgccctgatcatcctgccggacttcaacaccgccgccctgagcttcgacccgcacgccatcggcccgctgcagctggtgggcctggccgtgctgctggccggcctggccttcagcgtgtgggcccgcctgcacctgggcaccaactggagcgtgagcgtgaccctgaaggaggaccacgagctggtgcgcagcggcccgtacggcctggtgcgccacccgatctacaccggctgcctgctggccctgctgggcgccgtgctgatcggcgcccagtggcgcggcgtggccggcctggtgctgatcttcgccagcctggcctacaaggtgcgcgtggaggagagctggctgaccggccacttcggccgcgcctacagccagtaccgccgcgacgtggccgccctgatcccgggcctgttctgagcgacctgaatggcgaaagtcatcaataaaacgaaaggctcagtcgaaagactgggcctttcgttttataagctt

SEQ ID NO: 17 - DNA, 5062 bp, synthetic construct
  misc_feature 1..5062: note = pPG7 insert
  source 1..5062: mol_type = other DNA
ggtacccttggactcctgttgatagatccagtaatgacctcagaactccatctggatttgttcagaacgctcggttgccgccgggcgttttttattggtgagaatccagagctcttgacaaggtcggaaaattgaagtataatatcagtagggggttctagagatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgcgggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgcagcgcctgaaagccagcaaccatcagcacatggcttcgctggccaccggcgaatacgagcaggcgcgctttcacatcgaccaggtgctggcgctcaacgatcggggctacttggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggcagcggtacgcgtgcaccgcgccctgctcgagcacctgctgaagcaaccggagccgcagcgggcggaacgcgcggcgggattccacgagatggccatcggctacgcctgaacccttgacctgcgaagacccggcctggccgggctttgcggttgcataacgcacggagtagccccatgcacgccatcctcatcgccatcggctcggccggcgacgtatttcccttcatcggcctggcccggaccctgaaactgcgcgggcaccgcgtgagcctctgcaccatcccggtgtttcgcgacgcggtggagcagcacggcatcgcgttcgtcccgctgagcgacgaactgacctaccgccggaccatgggcgatccgcgcctgtgggaccccaagacgtccttcggcgtgctctggcaagccatcgccgggatgatcgagccggtctacgagtacgtctcggcgcagcgccatgacgacatcgtggtggtcggctcgctatgggcgctgggcgcacgcatcgctcacgagaagtacgggattccctacctgtccgcgcaggtctcgccatcgaccctgttgtcggcgcacctgccgccggtacaccccaagttcaacgtgcccgagcagatgccgctggcgatgcgcaagctgctctggcgctgcatcgagcgcttcaagctggatcgcacctgcgcgccggagatcaacgcggtgcgccgcaaggtcggcctggaaacgccggtgaagcgcatcttcacccaatggatgcattcgccgcagggcgtggtctgcctgttcccggcctggttcgcgccgccccagcaggattggccgcaacccctgcacatgaccggcttcccgctgttcgacggcagtatcccggggaccccgctcgacgacgaactgcaacgctttctcgatcagggcagccggccgctggtgttcacccagggctcgaccgaacacctgcagggcgacttctacgccatggccctgcgcgcgctggaacgcctcggcgcgcgtgggatcttcctcaccggcgccggccaggaaccgctgcgcggcttgccgaaccacgtgctgcagcgcgcctacgcgccactgggagccttgctgccatcgtgcgccgggctggtccatccgggcggtatcggcgccatgagcctagccttggcggcgggggtgccgcaggtgctgctgccctgtgcccacgaccagttcgacaatgccgaacggctggtccggctcggctgcgggatgcgcctgggcgtgccgttgcgcgagcaggagttgcgcggggcgctgtggcgcttgctcgaggacccggccatggcggcggcctgtcggcgtttcatggaattgtcacaaccgcacagtatcgcttgcggtaaagcggcccaggtggtcgaacgttgtcatagggagggggatgctcgatggctgaaggctgcgtcctgaacggtgctggcataacagatagggttgccatgattttgccgtatcggcaaggctgcgcgcttgacagcgtcataccccgggccaattctgctgtgatgcattttatcgatcagggcttactgcaatggagttcccgatggcccagagcaacatcatcgccggcatggacctgaaccgcctggaccgcatcgccgagcacctggaccgcgcctacctgcacccgggcaagctggccggcaccatgaccctggtggcccgccgcggcgaggtggtgtactgccaggcccagggcctgcgcgacgtggagcgccagctgccggtggagcgcgacaccctgttccgcatctacagcatgaccaagccgatcaccagcatcgccctgatgcagctgtacgagcagggccgcttcctgctggacgagccggtgcacaagtacatcccgacctggaagaacctgcgcgtgtacaagaccggcagccacccgcagatgctgaccaccgccccgcagcgcccgatgaccatccgcgacctgctgacccaccagagcggcctgacctacggcttcatgaaccgcaccaacgtggacgccgcctaccgcagcctgaagctggacggcggcccgggccacaccctggaccgcctgatcgacgagctggcccgcctgccgctggagttcagcccgggcaccgcctggaactacagcgtggccaccgacgtgtgcggctacctggtgcagctgctgagcggcatgagcctggacgactacttcagcaagcacatcttccagccgctgggcatgccggacaccttcttcaccgtgccggccgagaagctgagccgcttcgccgcctgctacgagtaccagccgggcgacagcttcagcctgcaggacgacccgcagggcagcgccttcgccaaggcccacggctacctgagcggcggcggcggcctggtgagctgcgtggacgactactaccgcttcgcccaggccctggccaacggcggcgagctggacggcgcccgcatcatcggccgcaagaccctggagttcatgcgcatgaaccacctgccggacaacaagggcctgccggacgtggccatcggcagcttcagcgagaccccgtacgacggcaccggcttcggcctgggcttcagcgtgaagctggacgtggccaagagccagaccgtgggcagcgtgggcgagtacggctggggcggcatggccagcaccaacttcttcatcgacccggaggaggacctgctgatggtgttcatgacccagctgatcccgagcagcacctacgccgtgcgccaggagctgcgcgccatcatcaacggcgccctggtggactgaagcgcagggcgcgccggtcggcgcgataaaacgaaaggctcagtcgaaagactgggcctttcgttttatcgggccccccctcgaggtcgacggtatcgatacttggactcctgttgatagatccagtaatgacctcagaactccatctggatttgttcagaacgctcggttgccgccgggcgttttttattggtgagaatccagagctcttgacaaggtcggaaaattgaagtataatatcagtagggggttctagagatggaccggatagacatgggcgtgctggtggtactgttcaatcctggcgacgacgacctggaacaccttggcgaactggcggcggcgtttccgcaactgcgcttccttgccgtcgacaactcaccgcacagcgatccgcagcgcaatgcccggctgcgcgggcaaggcatcgccgtgctgcaccacggcaaccggcagggcatcgccggcgccttcaaccagggactcgacgcgctattccggcgtggcgtgcagggtgtgctgctgctcgaccaggactcccgtcccggcggcgccttcctcgccgcccagtggcgcaacctgcaggcgcgcaacggtcaggcctgcctgctcggcccacggatcttcgaccggggtgaccggcgcttcctgccggccatccatctcgacggactgacgctcaggcaattgtctctggacggcctgacgaccccgcagcgcacctcgttcctgatctcctccggctgcctgctgacccgcgaggcctaccagcgcctcggccacttcgacgaggaactgttcatcgaccacgtggacaccgaatacagcctgcgcgcccaggcgctggacgtgcccctgtacgtcgacccgcggctggtcctcgagcaccgcatcggcacgcgcaagacccgccgcctcggcggtctcagcctcagcgcgatgaaccacgccccgctgcgccgctactacctggcgcgcaacggcctgctggtcctgcgccgctacgcccggtcctcgccgctggccctgctggcgaacctgccgaccctgacccagggcctcgcggtgctcctgctcgaacgcgacaagctgctcaagctgcgctgcctgggctggggcctgtgggacggcctgcggggacgcggcggcgcgctggagaccaaccgcccgcgcctgctgaagcgcctcgccggcccggccgtggcgtccgtagcttccggcaaggccaaggcctagtcggcgaaacgcattcccgcataggataaaacgaaaggctcagtcgaaagactgggcctttcgttttataagctt

SEQ ID NO: 18 - DNA, 5696 bp, synthetic construct
  misc_feature 1..5696: note = pPG8 insert
  source 1..5696: mol_type = other DNA
ggtacccttggactcctgttgatagatccagtaatgacctcagaactccatctggatttgttcagaacgctcggttgccgccgggcgttttttattggtgagaatccagagctcttgacaaggtcggaaaattgaagtataatatcagtagggggttctagagatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgcgggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgcagcgcctgaaagccagcaaccatcagcacatggcttcgctggccaccggcgaatacgagcaggcgcgctttcacatcgaccaggtgctggcgctcaacgatcggggctacttggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggcagcggtacgcgtgcaccgcgccctgctcgagcacctgctgaagcaaccggagccgcagcgggcggaacgcgcggcgggattccacgagatggccatcggctacgcctgaacccttgacctgcgaagacccggcctggccgggctttgcggttgcataacgcacggagtagccccatgcacgccatcctcatcgccatcggctcggccggcgacgtatttcccttcatcggcctggcccggaccctgaaactgcgcgggcaccgcgtgagcctctgcaccatcccggtgtttcgcgacgcggtggagcagcacggcatcgcgttcgtcccgctgagcgacgaactgacctaccgccggaccatgggcgatccgcgcctgtgggaccccaagacgtccttcggcgtgctctggcaagccatcgccgggatgatcgagccggtctacgagtacgtctcggcgcagcgccatgacgacatcgtggtggtcggctcgctatgggcgctgggcgcacgcatcgctcacgagaagtacgggattccctacctgtccgcgcaggtctcgccatcgaccctgttgtcggcgcacctgccgccggtacaccccaagttcaacgtgcccgagcagatgccgctggcgatgcgcaagctgctctggcgctgcatcgagcgcttcaagctggatcgcacctgcgcgccggagatcaacgcggtgcgccgcaaggtcggcctggaaacgccggtgaagcgcatcttcacccaatggatgcattcgccgcagggcgtggtctgcctgttcccggcctggttcgcgccgccccagcaggattggccgcaacccctgcacatgaccggcttcccgctgttcgacggcagtatcccggggaccccgctcgacgacgaactgcaacgctttctcgatcagggcagccggccgctggtgttcacccagggctcgaccgaacacctgcagggcgacttctacgccatggccctgcgcgcgctggaacgcctcggcgcgcgtgggatcttcctcaccggcgccggccaggaaccgctgcgcggcttgccgaaccacgtgctgcagcgcgcctacgcgccactgggagccttgctgccatcgtgcgccgggctggtccatccgggcggtatcggcgccatgagcctagccttggcggcgggggtgccgcaggtgctgctgccctgtgcccacgaccagttcgacaatgccgaacggctggtccggctcggctgcgggatgcgcctgggcgtgccgttgcgcgagcaggagttgcgcggggcgctgtggcgcttgctcgaggacccggccatggcggcggcctgtcggcgtttcatggaattgtcacaaccgcacagtatcgcttgcggtaaagcggcccaggtggtcgaacgttgtcatagggagggggatgctcgatggctgaaggctgcgtcctgaacggtgctggcataacagatagggttgccatgattttgccgtatcggcaaggctgcgcgcttgacagcgtcataccccgggccaattctgctgtgatgcattttatcgatcagggcttactgcaatggagttcccgatggcccagagcaacatcatcgccggcatggacctgaaccgcctggaccgcatcgccgagcacctggaccgcgcctacctgcacccgggcaagctggccggcaccatgaccctggtggcccgccgcggcgaggtggtgtactgccaggcccagggcctgcgcgacgtggagcgccagctgccggtggagcgcgacaccctgttccgcatctacagcatgaccaagccgatcaccagcatcgccctgatgcagctgtacgagcagggccgcttcctgctggacgagccggtgcacaagtacatcccgacctggaagaacctgcgcgtgtacaagaccggcagccacccgcagatgctgaccaccgccccgcagcgcccgatgaccatccgcgacctgctgacccaccagagcggcctgacctacggcttcatgaaccgcaccaacgtggacgccgcctaccgcagcctgaagctggacggcggcccgggccacaccctggaccgcctgatcgacgagctggcccgcctgccgctggagttcagcccgggcaccgcctggaactacagcgtggccaccgacgtgtgcggctacctggtgcagctgctgagcggcatgagcctggacgactacttcagcaagcacatcttccagccgctgggcatgccggacaccttcttcaccgtgccggccgagaagctgagccgcttcgccgcctgctacgagtaccagccgggcgacagcttcagcctgcaggacgacccgcagggcagcgccttcgccaaggcccacggctacctgagcggcggcggcggcctggtgagctgcgtggacgactactaccgcttcgcccaggccctggccaacggcggcgagctggacggcgcccgcatcatcggccgcaagaccctggagttcatgcgcatgaaccacctgccggacaacaagggcctgccggacgtggccatcggcagcttcagcgagaccccgtacgacggcaccggcttcggcctgggcttcagcgtgaagctggacgtggccaagagccagaccgtgggcagcgtgggcgagtacggctggggcggcatggccagcaccaacttcttcatcgacccggaggaggacctgctgatggtgttcatgacccagctgatcccgagcagcacctacgccgtgcgccaggagctgcgcgccatcatcaacggcgccctggtggactgaagcgcagggcgcgccggtcggcgcgataaaacgaaaggctcagtcgaaagactgggcctttcgttttatcgggccccccctcgaggtcgacggtatcgatacttggactcctgttgatagatccagtaatgacctcagaactccatctggatttgttcagaacgctcggttgccgccgggcgttttttattggtgagaatccagagctcttgacaaggtcggaaaattgaagtataatatcagtagggggttctagagatggaccggatagacatgggcgtgctggtggtactgttcaatcctggcgacgacgacctggaacaccttggcgaactggcggcggcgtttccgcaactgcgcttccttgccgtcgacaactcaccgcacagcgatccgcagcgcaatgcccggctgcgcgggcaaggcatcgccgtgctgcaccacggcaaccggcagggcatcgccggcgccttcaaccagggactcgacgcgctattccggcgtggcgtgcagggtgtgctgctgctcgaccaggactcccgtcccggcggcgccttcctcgccgcccagtggcgcaacctgcaggcgcgcaacggtcaggcctgcctgctcggcccacggatcttcgaccggggtgaccggcgcttcctgccggccatccatctcgacggactgacgctcaggcaattgtctctggacggcctgacgaccccgcagcgcacctcgttcctgatctcctccggctgcctgctgacccgcgaggcctaccagcgcctcggccacttcgacgaggaactgttcatcgaccacgtggacaccgaatacagcctgcgcgcccaggcgctggacgtgcccctgtacgtcgacccgcggctggtcctcgagcaccgcatcggcacgcgcaagacccgccgcctcggcggtctcagcctcagcgcgatgaaccacgccccgctgcgccgctactacctggcgcgcaacggcctgctggtcctgcgccgctacgcccggtcctcgccgctggccctgctggcgaacctgccgaccctgacccagggcctcgcggtgctcctgctcgaacgcgacaagctgctcaagctgcgctgcctgggctggggcctgtgggacggcctgcggggacgcggcggcgcgctggagaccaaccgcccgcgcctgctgaagcgcctcgccggcccggccgtggcgtccgtagcttccggcaaggccaaggcctagtcggcgaaacgcattcccgcataggtggaaattccataggttgatcaaagagggggttctagagatgaacatcgcccagagcatcgccatcgtggtgccgtggaccctgtggctgctgtactggatcgccaccagcaaccgcgtgaaggagaccgcccgcaaggaggccagcctgagccgcaccctgcagagcatcccgctgatcggcggcggcgccctgatcatcctgccggacttcaacaccgccgccctgagcttcgacccgcacgccatcggcccgctgcagctggtgggcctggccgtgctgctggccggcctggccttcagcgtgtgggcccgcctgcacctgggcaccaactggagcgtgagcgtgaccctgaaggaggaccacgagctggtgcgcagcggcccgtacggcctggtgcgccacccgatctacaccggctgcctgctggccctgctgggcgccgtgctgatcggcgcccagtggcgcggcgtggccggcctggtgctgatcttcgccagcctggcctacaaggtgcgcgtggaggagagctggctgaccggccacttcggccgcgcctacagccagtaccgccgcgacgtggccgccctgatcccgggcctgttctgagcgacctgaatggcgaaagtcatcaataaaacgaaaggctcagtcgaaagactgggcctttcgttttataagctt

SEQ ID NO: 19 - DNA, 2472 bp, synthetic construct
  source 1..2472: mol_type = other DNA
ggtacccttggactcctgttgatagatccagtaatgacctcagaactccatctggatttgttcagaacgctcggttgccgccgggcgttttttattggtgagaatccagagctcttgacaaggtcggaaaattgaagtataatatcagtagggggttctagagatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgcgggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgcagcgcctgaaagccagcaaccatcagcacatggcttcgctggccaccggcgaatacgagcaggcgcgctttcacatcgaccaggtgctggcgctcaacgatcggggctacttggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggcagcggtacgcgtgcaccgcgccctgctcgagcacctgctgaagcaaccggagccgcagcgggcggaacgcgcggcgggattccacgagatggccatcggctacgcctgaacccttgacctgcgaagacccggcctggccgggctttgcggttgcataacgcacggagtagccccatgcacgccatcctcatcgccatcggctcggccggcgacgtatttcccttcatcggcctggcccggaccctgaaactgcgcgggcaccgcgtgagcctctgcaccatcccggtgtttcgcgacgcggtggagcagcacggcatcgcgttcgtcccgctgagcgacgaactgacctaccgccggaccatgggcgatccgcgcctgtgggaccccaagacgtccttcggcgtgctctggcaagccatcgccgggatgatcgagccggtctacgagtacgtctcggcgcagcgccatgacgacatcgtggtggtcggctcgctatgggcgctgggcgcacgcatcgctcacgagaagtacgggattccctacctgtccgcgcaggtctcgccatcgaccctgttgtcggcgcacctgccgccggtacaccccaagttcaacgtgcccgagcagatgccgctggcgatgcgcaagctgctctggcgctgcatcgagcgcttcaagctggatcgcacctgcgcgccggagatcaacgcggtgcgccgcaaggtcggcctggaaacgccggtgaagcgcatcttcacccaatggatgcattcgccgcagggcgtggtctgcctgttcccggcctggttcgcgccgccccagcaggattggccgcaacccctgcacatgaccggcttcccgctgttcgacggcagtatcccggggaccccgctcgacgacgaactgcaacgctttctcgatcagggcagccggccgctggtgttcacccagggctcgaccgaacacctgcagggcgacttctacgccatggccctgcgcgcgctggaacgcctcggcgcgcgtgggatcttcctcaccggcgccggccaggaaccgctgcgcggcttgccgaaccacgtgctgcagcgcgcctacgcgccactgggagccttgctgccatcgtgcgccgggctggtccatccgggcggtatcggcgccatgagcctagccttggcggcgggggtgccgcaggtgctgctgccctgtgcccacgaccagttcgacaatgccgaacggctggtccggctcggctgcgggatgcgcctgggcgtgccgttgcgcgagcaggagttgcgcggggcgctgtggcgcttgctcgaggacccggccatggcggcggcctgtcggcgtttcatggaattgtcacaaccgcacagtatcgcttgcggtaaagcggcccaggtggtcgaacgttgtcatagggagggggatgctcgatggctgaaggctgcgtcctgaacggtgctggcataacagatagggtataaaacgaaaggctcagtcgaaagactgggcctttcgttttataagctt

SEQ ID NO: 20 - DNA, 570 bp, unidentified
  source 1..570: mol_type = other DNA
atgaatatcgctcagagcatcgctatcgtcgtaccgtggacgctgtggctgctgtattggatcgcgacgtcgaaccgcgtcaaggaaaccgcgcgcaaggaggcgtctctgtcgcgcacgttgcagtcgattccgttgatcggcggaggcgcactgatcatcctgccggacttcaacacagcggcgttgtccttcgatccgcatgcgatcgggccgctgcagctggtgggcctggccgtgctgttagctggacttgcgttttccgtgtgggcgcggctgcatctcggcaccaactggagcgtctcggtcacgctgaaggaagaccacgagctggtgcgcagcggaccctatggactggttcgtcatccgatctataccggatgcctgctggcgcttctcggtgctgtgctgatcggcgcgcaatggcgtggcgtggctgggctagtgctgattttcgcctcgctggcgtacaaggtccgggtcgaggagagttggctgacgggtcacttcggccgtgcgtattcgcagtatcgccgcgacgtcgccgcgctgattcccggtctcttctga

SEQ ID NO: 21 - DNA, 1230 bp, unidentified; identical to SEQ ID NO: 13
  source 1..1230: mol_type = other DNA

(19) United States
(12) Patent Application Publication
(10) Pub. No.: US 2025/0333772 A1
(43) Pub. Date: Oct. 30, 2025

(54) METHOD OF MAKING GLYCOMONOLIPIDS

(71) Applicant: The Procter & Gamble Company, Cincinnati, OH (US)

(72) Inventors: Jose Carlos GARCIA-GARCIA, Cincinnati, OH (US); Dakota James BROCK, West Chester, OH (US); Hilda Andamiche NAMANJA-MAGLIANO, Loveland, OH (US); Zubin Sarosh KHAMBATTA, Fairfield, OH (US); Tomislav TICAK, Mason, OH (US); Lijuan LI, Lebanon, OH (US)

(21) Appl. No.: 19/062,630

(22) Filed: Feb. 25, 2025

Related U.S. Application Data
(60) Provisional application No. 63/557,812, filed on Feb. 26, 2024.
(60) Provisional application No. 63/558,670, filed on Feb. 28, 2024.
(60) Provisional application No. 63/641,630, filed on May 2, 2024.

(51) Int. Cl. C12P 19/02; C12N 9/10; C12N 9/18; C12N 15/70

(52) U.S. Cl. CPC C12P 19/02; C12N 9/1029; C12N 9/18; C12N 15/70; C12Y 203/01; C12Y 301/01001

(57) ABSTRACT

A method of making glycomonolipids; said method comprising the following steps: a) reacting glycodilipids with a hydrolase to form glycomonolipids; and b) optionally isolating the glycomonolipids.

### CROSS REFERENCE TO RELATED APPLICATIONS

This application claims the benefit, under 35 U.S.C. § 119(e), to U.S. Provisional Application Nos. 63/641630 filed May 2, 2024, 63/558,670 filed Feb. 28, 2024, and 63/557,812, filed Feb. 26, 2024, the entire disclosure of which is fully incorporated by reference herein.

### REFERENCE TO A SEQUENCE LISTING

The sequence information provided in the Sequence Listing XML file named “16700M sequence listing final”, created on Feb. 24, 2025 and with a file size of 59,619 bytes, is hereby incorporated by reference into this application.

### FIELD OF THE INVENTION

The present invention relates to cells and methods for producing glycomonolipids, and more particularly relates to cells and methods for producing rhamnomonolipids.

### BACKGROUND OF THE INVENTION

In personal care products, surfactants may be used for cleansing, foaming, thickening, emulsifying, solubilizing, and antimicrobial effects, among other uses. But many surfactants that are commonly used are made from starting materials such as petrochemicals, while consumers are seeking more natural and milder materials in their products. Biosurfactants, which are surfactants of microbial origin, can provide improved efficacy without performance trade-offs.

### SUMMARY OF THE INVENTION

A method of making glycomonolipids; said method comprising the following steps: a. reacting glycodilipids with a hydrolase to form glycomonolipids; and b. optionally isolating the glycomonolipids; wherein the glycomonolipids have the following Formula 1:

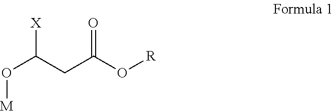

wherein X is selected from an alkyl, aryl, heteroalkyl, heteroaryl, unsaturated alkenyl, and unsaturated heteroalkenyl; and has a carbon chain length from 4 to 22;
wherein M is selected from one or two of glucose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl, and all possible stereoisomers thereof, and all combinations thereof.

### BRIEF DESCRIPTION OF THE FIGURES

For a more complete understanding of the disclosure, reference should be made to the following detailed description and drawing Figures.

FIG. 1 is a map for pPG1 insert;

FIG. 2 is a map for pPG2 insert;

FIG. 3 is a map for pPG6 insert;

FIG. 4 is a map for pPG7 insert;

FIG. 5 is a map for pPG8 insert;

FIG. 6 is an alignment of a codon-optimized rhlM CDS from “SAMNO5192563_1007175” (SEQ ID NO: 4) with the original sequence (SEQ ID NO: 20); and

FIG. 7 is an alignment of a codon-optimized eh7 CDS from “KY483644.1” (SEQ ID NO: 5) with the original sequence (SEQ ID NO: 21).

FIG. 8 shows exemplary rhamnolipid structures.

FIG. 9A is a polyacrylamide gel stained with Imperial protein stain to visualize the presence and absence of recombinant hydrolase following isolation of recombinant hydrolase from reactant solution.

FIG. 9B is a polyacrylamide gel stained with Imperial protein stain to visualize the presence and absence of recombinant hydrolase following recombinant hydrolase recovery and reuse.

FIG. 10 is a graph of Mean(Area) vs. Analyte for constructs shown in FIGS. 1 and 2 when expressed via fermentation.

FIG. 11 is graph showing the sum of the detected areas under the peak of all mono-rhamno-di-lipid congeners of various chain lengths (“RCC-sum”) and the sum of all di-rhamno-mono-lipid congeners (“RRC-sum”) of various chain lengths. Pseudomonas putida expressing RhlC was able to accumulate di-rhamno-mono-lipids (RRC, green), while the strain harboring plasmid pPG2, without RhlC, was not.

FIG. 12 shows P. putida expressing rhlM (SEQ ID NO: 4) to produce methylated Rha-C10, RhaRha-C10, and Rha-C10C10 (for example) where these (and other) congeners were not observed in the absence of rhlM.

FIG. 13 is the production of mono-rhamno-mono-lipid using Fatty Acid Distillate as feedstock.

FIG. 14 is a graph of spectra of absorbance as a function of wavelength for Rheance One and Inventive Example B.

### DETAILED DESCRIPTION OF THE INVENTION

There is interest in non-traditional surfactants, such as glycolipid biosurfactants, as many palm- and petroleum-based surfactants suffer from sustainability, environmental, and socioeconomic challenges. Glycolipids consist of a diverse group of naturally occurring surfactant molecules with a range of structures (made up of a sugar polar group and a lipid group). The two main commercial classes of glycolipids are rhamnolipids (produced via bacterial plus fermentation) and sophorolipids (produced via yeast fermentation of mixed oil & sugar feed). In addition to being seen as environmentally friendly chemicals and enabling green credentialling, these materials have many other potential benefits such as mildness, moisturization, and cleaning effectiveness.

Many current commercial glycolipids have poor performance and high costs compared to current surfactants. Knowing the structures of certain high-performing rhamnolipids, the present inventors sought to produce optimized glycolipids. The inventors hypothesized that changing the molecular structure via simplification of the surfactant headgroup and elongation to a single chain length (making them more structurally similar to typical surfactants) would increase surfactancy. One of the production strategies involved a short-term, semi-synthetic fermentation approach, in which commercial rhamnolipids were leveraged as the feedstock and hydrolyzed to produce simplified mono-lipid glycolipid congener structures, (as described in U.S. Patent No. 63/558,670, Attorney Docket No. 16682P2, which is incorporated by reference herein).

The present invention involves methods of making biosurfactants from glycodilipids. The biosurfactants may be made through use of a hydrolase to produce glycomonolipids. In some embodiments, commercial rhamnolipids may be used as the starting material. The commercial rhamnolipids, such as Rheance® One, sold by Evonik Industries AG, Essen, Germany, or others sold by BioReNuva, Austin TX, USA, and by Wanhua Chemical Group Co., Ltd., Yantai, China, may be hydrolyzed by a carboxylic ester hydrolase. The predominantly rhamnodilipids are hydrolyzed, resulting in predominantly rhamnomonolipids. In some embodiments, a carboxylic ester hydrolase may be engineered through recombinant DNA technology, such as by heterologous expression in a host cell. All of the inventive methods result in producing glycomonolipids [Formula 1], which offer commercial and consumer benefits.

The present invention provides methods and cells for making glycomonolipids. In certain embodiments, the method for producing glycomonolipids in a cell includes expressing in the cell one or more recombinant polypeptides that catalyze the conversion of glycodilipids with a hydrolase, in some embodiments a carboxylic ester hydrolase, and culturing the cell under conditions suitable for producing the polypeptide, such that glycomonolipids, in some embodiments rhamnomonolipids, are produced.

In certain embodiments, a method for producing glycomonolipids in a cell is provided, the method including expressing in the cell a polypeptide that has hydrolase activity; and culturing the cell under conditions suitable for producing the polypeptide, such that monorhamnolipids are produced.

Further provided is a method for producing methylated glycomonolipids in a cell, the method including expressing in the cell a polypeptide that has S-isoprenylcysteine O-methyltransferase (RhlM) activity; expressing in a cell a polypeptide that has S-isoprenylcysteine O-methyltransferase activity; and culturing the cell under conditions suitable for producing the polypeptides, such that methylated glycomonolipids are produced.

Also provided is a method for producing glycomonolipids in a cell, the rhamnomonolipids having a chain length from about 4 to 22 carbons. The method includes modifying the cell to increase carbon flow and culturing the cell under conditions suitable for carbon flow to be increased, such that glycomonolipids having a chain length from about 4 to about 22 carbons are produced.

Further provided is a Pseudomonas cell that produces rhamnomonolipids having a chain length from about 4 to 22 carbons.

The present invention provides a method of making glycomonolipids; said method comprising the following steps:
a. reacting glycodilipids with a hydrolase to form glycomonolipids; and
b. optionally isolating the glycomonolipids;
wherein the glycomonolipids have the following formula 1:

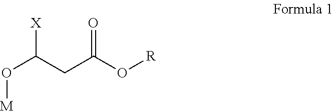

wherein X is selected from an alkyl, aryl, heteroalkyl, heteroaryl, unsaturated alkenyl, and unsaturated heteroalkenyl; and has a carbon chain length from 4 to 22;
   wherein M is selected from one or two of glucose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl, and all possible stereoisomers thereof.

In some embodiments, the hydrolase comprises a polypeptide sequence having at least 70% identity, in some cases 90% identity, to SEQ ID NO: 11 or fragments thereof.

In some embodiments, the hydrolase may be, but is not limited to, a carboxylic ester hydrolase selected from the group consisting of esterases (EC 3.1.1.1, EC 3.1.1.43, EC 3.1.1.84, EC 3.1.1.85, EC 3.1.1.86, EC 3.1.1.87, EC 3.1.1.112, EC 3.1.1.113, EC 3.1.1.114), lipases (EC 3.1.1.3, EC 3.1.1.23), phospholipase (EC 3.1.1.4), lysophospholipase (EC 3.1.1.5), acetylesterase (EC 3.1.1.6), depolymerase (EC 3.1.1.75, EC 3.1.1.76) cutinase (EC 3.1.1.74) and hydrolases (EC 3.1.1.22, EC 3.1.1.50, EC 3.1.1.71, EC 3.1.1.101, EC 3.1.1.102). The Enzyme Commission number (EC number) is a numerical classification scheme for enzymes, based on the chemical reactions they catalyze.

In some embodiments, the hydrolase may be encapsulated and/or immobilized.

In some embodiments, the hydrolase that is reacted with the glycodilipids may be made by being heterologously expressed in a host cell. The host cell may be a eukaryotic and/or a prokaryotic organism. The host cell may be, but is not limited to, at least one of Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Pseudomonas putida, Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis, Tetragenococcus koreensis, Methylobacterium sp., and Methylorubrum sp.

In some embodiments, the glycodilipids that are reacted with a hydrolase may be rhamnodilipids. In some embodiments, the resulting glycomonolipids may be rhamnomonolipids. For example, a starting glycodilipid may be commercially available Rheance One, sold by Evonik, which is a mixture of rhamnolipids that are predominantly rhamnodilipids (See Table 1.3b). In some embodiments, the starting glycodilipids may be rhamnodilipids, and the rhamnodilipids may be selected from, but not limited to, Rha-C8C8, Rha-C8C10, Rha-C8C10:1, Rha-C8C12, Rha-C9C10, Rha-C10:1C8, Rha-C10:1C10, Rha-C10C8, Rha-C10C9, Rha-C10C10, Rha-C10C10:1, Rha-C10C11, Rha-C10C12, Rha-C10C12:1, Rha-C10C14, Rha-C10C14:1, Rha-C10C16, Rha-C11C10, Rha-C12:1C8, Rha-C12:1C10, Rha-C12:1C12Rha-C12:1C12:1, Rha-C12C8, Rha-C12C10, Rha-C12C12, Rha-C12C12:1, Rha-C12C14:1, Rha-C14:1C10, RhaRha-C8C8, RhaRha-C8C10, RhaRha-C8C12, RhaRha-C8C12:1, RhaRha-C10:1C10, RhaRha-C10:1C12:1, RhaRha-C10C8, RhaRha-C10C10, RhaRha-C10C10:1, RhaRha-C10C12, RhaRha-C10C12:1, RhaRha-C10C14, RhaRha-C10C14:1, RhaRha-C12:1C8, RhaRha-C12:1C10, RhaRha-C12:1C12, RhaRha-C12:1C12:1, RhaRha-C12C8, RhaRha-C12C10, RhaRha-C12C12, RhaRha-C12C12:1, RhaRha-C12C14, RhaRha-C14:1C10, RhaRha-C14C10, and combinations thereof.

In some embodiments, a mixture of predominantly glycodilipids (which may be at least about 50% glycodilipids, at least about 70% glycodilipids, at least about 90% glycodilipids, by weight of the total starting material) may be reacted with a hydrolase. The result of the reaction may be a mixture of glycolipids that are predominantly glycomonolipids, for example, at least about 50 wt. %, at least about 70 wt. %, at least about 90 wt. %, or at least about 95 wt. % glycomonolipids (see Table 1.3b). After the reaction, the catalytic hydrolase may be isolated from the glycolipids and/or from the glycomonolipids; the glycomonolipids, which make up the majority of the glycolipids, may be isolated and/or purified from unreacted or unconverted glycodilipids. The total result of the inventive reaction may be, by weight, at least 50%, 70%, 90%, 95%, or 98% glycomonolipids.

In some embodiments, the glycodilipids, or rhamnodilipids, may be reacted with a hydrolase under certain conditions, such as, for example, at a pH from 6 to 10; at a temperature from 25° C. to 42° C.; and/or under agitation in a buffered solution.

In some embodiments, the resulting glycomonolipids or rhamnomonolipids do not exhibit detectable discoloration from the starting glycodilipids. Similarly, resulting rhamnomonolipids may not differ in color from the starting rhamnodilipids. Also, in some embodiments, the resulting glycomonolipids (or rhamnomonolipids) may have no difference in odor from the starting glycodilipids (or rhamnodilipids).

In some embodiments, the present invention provides a recombinant cell comprising the following:
(i) an enzyme (A) comprising the SEQ ID NO: 7;
(ii) an enzyme (B) comprising the SEQ ID NO: 8; and
(iii) an ester hydrolase comprising SEQ ID NO: 11.

In some embodiments of the recombinant cell, enzyme (A) may catalyze the conversion of a 3-OH fatty acid to a 3-(Hydroxyalkanoyloxy)alkanoic acid and enzyme (B) may catalyze the addition of a single rhamnose unit to 3-(Hydroxyalkanoyloxy)alkanoic acid.

In some embodiments of the recombinant cell, the recombinant cell may be selected from at least one of Pseudomonas putida, Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis Tetragenococcus koreensis, Methylobacterium sp., and Methylorubrum sp.

In some embodiments of the recombinant cell, the ester hydrolase may comprise a polypeptide sequence having at least 70% identity, in some embodiments at least 80%, at least 90%, or at least 95% identity to SEQ ID NO: 11 or fragments thereof. And in still other embodiments, the recombinant cell may additionally comprise an enzyme (C) comprising at least 70% identity, in some embodiments at least 80%, at least 90%, or at least 95% identity to SEQ ID NO: 9, or fragments thereof.

In other embodiments, the present invention provides a method of making rhamnomonolipids in a recombinant cell, said method comprising the following steps:
a. culturing the recombinant cell to generate rhamnomonolipids; and
b. optionally isolating the resulting rhamnomonolipids;
wherein said recombinant cell comprises:
   (i) an enzyme (A) comprising the SEQ ID NO: 7;
   (ii) an enzyme (B) comprising the SEQ ID NO: 8; and
   (iii) an ester hydrolase comprising SEQ ID NO: 11.

In some embodiments of the method of making rhamnomonolipids in a recombinant cell, enzyme (A) may catalyze the conversion of 3-OH fatty acid to 3-(Hydroxyalkanoyloxy)alkanoic acid and enzyme (B) may catalyze the addition of a single rhamnose unit to 3-(Hydroxyalkanoyloxy)alkanoic acid. In some embodiments of the method, the recombinant cell is selected from at least one of Pseudomonas putida, Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis Tetragenococcus koreensis, Methylobacterium sp., and Methylorubrum sp.

In some embodiments of the method of making rhamnomonolipids in a recombinant cell, the ester hydrolase may comprise a polypeptide sequence having at least 70% identity to SEQ ID NO: 11 or fragments thereof, in some embodiments at least 80% identity, and in some embodiments at least 90% identity.

In some embodiments of the method, the recombinant cell may additionally comprise an enzyme (C) comprising SEQ ID NO: 9. In some embodiments of the method, the recombinant cell may further comprise a methyltransferase having a sequence having at least 70% identity to SEQ ID NO: 10 or fragments thereof.

In some embodiments of the method, the making of rhamnolipids may be done by culturing the recombinant cell at a pH from 6 to 10; at a temperature from 25° C. to 42° C.; and/or under agitation in a buffered solution with a carbon feedstock; the feedstock may be selected from fatty acid distillate, used soybean oil, soybean oil soapstock, orange peels, distillery waste, wheat straw, sweet water, sugarcane begasse, a cellulosic waste stream, and combinations thereof.

In some embodiments, the present invention provides a recombinant cell comprising the following:
(i) an enzyme (A) comprising SEQ ID NO: 7;
(ii) an enzyme (B) comprising SEQ ID NO: 8; and
(iii) a methyltransferase comprising SEQ ID NO: 10.

In some embodiments, the methyltransferase comprising SEQ ID NO: 10 may catalyze methylating rhamnolipids.

In some embodiments of the present invention, a method is provided of producing rhamnomonolipids in a recombinant cell, said method comprising the following steps:
a. culturing the cell to generate rhamnomonolipids;
b. optionally isolating the resulting rhamnomonolipids;
wherein said recombinant cell comprises:
   (i) an enzyme (A) comprising the SEQ ID NO: 7;
   (ii) an enzyme (B) comprising the SEQ ID NO: 8; and
   (iii) a methyltransferase comprising SEQ ID NO: 10 that catalyzes methylating rhamnolipids.

In some embodiments, the methyltransferase may have a sequence having at least 70% identity to SEQ ID NO: 10 or fragments thereof.

In some embodiments, the present invention provides a method of producing glyco-monolipids, wherein glycomonolipids are generated through growth of an engineered cell by either:
A.) constitutive growth from a carbon source; (such as feedstocks or sole carbon sources) to yield glycomonolipids;
B.) grown cells from a carbon source; (such as feedstocks or sole carbon) are then lysed to generate a composite of proteins to react starting materials to lead to glycomonolipids in vitro or ex vivo;
comprising the following steps:
   a. reacting glycodilipids with a carboxylic ester hydrolase to form glycomonolipids; and
   b. optionally isolating the glycomonolipids;
   wherein the glycomonolipids have the following formula 1:

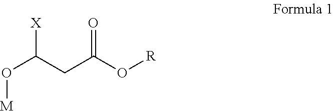

wherein X is selected from an alkyl, aryl, heteroalkyl, heteroaryl, unsaturated alkenyl, and unsaturated heteroalkenyl; and has a carbon chain length from 4 to 22;
wherein M is selected from one or two of glucose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl, and all possible stereoisomers thereof.

In some embodiments, a method is provided of growing a cell comprising recombinant polypeptides having esterase, lipase, phospholipase, lysopholipase, acetylesterase, pectinesterase, lactonase, tannase, cutinase and hydrolase activity, wherein the cell comprising the recombinant polypeptides produces more rhamnomonolipids than an otherwise similar cell that does not comprise the recombinant polypeptide.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

When used in the context of a chemical group: “hydrogen” means —H; “hydroxy” means —OH; “oxo” means ═O; “carbonyl” means —C(═O)—; “carboxy” and “carboxylate” mean —C(═O)OH (also written as —COOH or —CO2H) or a deprotonated form thereof; “amino” means —NH2; “hydroxyamino” means —NHOH; “nitro” means —NO2; “imino” means=NH; “amine oxide” means N⁺O⁻ where N has three covalent bonds to atoms other than 0; “hydroxamic” or “hydroxamate” means —C(O)NHOH or a deprotonated form thereof.

The term “cation” refers to an atom, molecule, or a chemical group with a net positive charge including single and multiple charged species. Cations can be individual atoms such as metals, non-limiting examples include Na⁺ or Ca⁺², individual molecules, non-limiting examples include (CH₃)₄N⁺, or a chemical group, non-limiting examples include-N(CH₃)₃+. The term “amine cation” refers to a particular molecular cation, of the form NR₄+ where the four substituting R moieties can be independently selected from H and alkyl, non-limiting examples include NH₄⁺ (ammonium), CH₃NH₃+(methylammonium), CH₃CH₂NH₃⁺ (ethylammonium), (CH₃)₂NH₂⁺ (dimethylammonium), (CH₃)₃NH⁺ (trimethyl ammonium), and (CH₃)₄N⁺ (tetramethylammonium). In some embodiments, a cation may be selected from Na+, K+, Li+, Cs+, +NH₃R2; +NH₂R2R3; +NHR2R3R4, +NR₂R3R4R5 wherein R2, R3, R4, and R5 are each independently selected from an alkyl, branched alkyl, and cyclic alkyl.

The term “anion” refers to an atom, molecule, or chemical group with a net negative charge including single and multiply charged species. Anions can be individual atoms, for example but not limited to halides F⁻, Cl⁻, Br⁻, individual molecules, non-limiting examples include CO₃⁻², H₂PO₄⁻, HPO₄⁻², PO₄⁻³, HSO₄⁻, SO₄⁻², or a chemical group, non-limiting examples include sulfate, phosphate, sulfonate, phosphonate, phosphinate, sulfonate, mercapto, carboxylate, amine oxide, hydroxamate and hydroxyl amino. Deprotonated forms of previously defined chemical groups are considered anionic groups if the removal of the proton results in a net negative charge. In solutions, chemical groups are capable of losing a proton and become anionic as a function of pH according to the Henderson-Hasselbach equation (pH=pKa+log₁₀([A⁻]/[HA]; where [HA] is the molar concentration of an undissociated acid and [A⁻] is the molar concentration of this acid's conjugate base). When the pH of the solution equals the pKa value of functional group, 50% of the functional group will be anionic, while the remaining 50% will have a proton. Typically, a functional group in solution can be considered anionic if the pH is at or above the pKa of the functional group.

The term “salt” or “salts” refers to the charge neutral combination of one or more anions and cations. For example, when R is denoted as a salt for the carboxylate group, —COOR, it is understood that the carboxylate (—COO—) is an anion with a negative charge −1, and that the R is a cation with a positive charge of +1 to form a charge neutral entity with one anion of charge −1, or R is a cation with a positive charge of +2 to form a charge neutral entity with two anions both of −1 charge.

The term “saturated” as used herein means the chemical compound or group so modified has no carbon-carbon double and no carbon-carbon triple bonds, except as noted below. In the case of substituted versions of saturated chemical groups, one or more carbon oxygen double bond or a carbon nitrogen double bond may be present. When such a bond is present, then carbon-carbon double bonds that may occur as part of keto-enol tautomerism or imine/enamine tautomerism are not precluded.

The term “aliphatic” when used without the “substituted” modifier signifies that the chemical compound/group so modified is an acyclic or cyclic, but non-aromatic hydrocarbon chemical compound or group. In aliphatic chemical compounds/groups, the carbon atoms can be joined together in straight chains, branched chains, or non-aromatic rings (alicyclic). Aliphatic chemical compounds/groups can be saturated, that is joined by single bonds (alkanes/alkyl), or unsaturated, with one or more double bonds (alkenes/alkenyl), or with one or more triple bonds (alkynes/alkynyl).

The term “alkyl” when used without the “substituted” modifier refers to a monovalent saturated aliphatic group with a carbon atom as the point of attachment, a linear or branched, cyclo, cyclic, or acyclic structure, and no atoms other than carbon and hydrogen. Thus, as used herein cycloalkyl is a subset of alkyl, with the carbon atom that forms the point of attachment also being a member of one or more non-aromatic ring structures wherein the cycloalkyl group consists of no atoms other than carbon and hydrogen. As used herein, the term does not preclude the presence of one or more alkyl groups (carbon number limitation permitting) attached to the ring or ring system. The groups —CH₃ (Me), —CH₂CH₃ (Et), —CH₂CH₂CH₃ (n-Pr or propyl), —CH(CH₃)₂(i-Pr, ′Pr, or isopropyl), —CH(CH₂)₂(cyclopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tertbutyl, t-butyl, t-Bu, or tBu), —CH₂C(CH₃)₃(neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. An “alkane” refers to the compound H—R, wherein R is alkyl as this term is defined above.

When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, —S(O)₂NH₂, —P(O)(OH)₂, —P(O)(OH)OP(O)(OH)₂, —OP(O)(OH)₂, —OP(O)(OH)OP(O)(OH)₂, —S(O)₂(OH), or —OS(O)₂(OH). The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, —CH₂CH₂C1, —CH₂P(O)(OH)₂, —CH₂P(O)(OH)OP(O)(OH)₂, —CH₂S(O)₂(OH), and —CH₂OS(O)₂(OH). The term “haloalkyl” is a subset of substituted alkyl, in which one or more hydrogen atoms has been substituted with a halo group and no other atoms aside from carbon, hydrogen and halogen are present. The group, —CH₂Cl is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups.

The term “alkenyl” when used without the “substituted” modifier refers to a monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —C(CH₃)═CH₂ (methyl-vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, >C═CH₂ (vinylidine), —CH═CH—, —CH═C(CH₃)CH₂—, and —CH═CHCH₂—, are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above.

The term “aryl” when used without the “substituted” modifier refers to a functional group derived from a simple aromatic ring compound where the point of attachment is a carbon atom on the aromatic ring. An aromatic ring is a hydrocarbon that has a cyclic structure and a delocalized electron system. An aryl group is formed by removing one hydrogen atom from the ring. The name of the aryl group is based on the name of the aromatic ring with the -yl suffix, such as phenyl, naphthyl, indolyl, etc. Aryl groups may be substituted with alkyl and/or heteroalkyl chains and may have one or more heteroatoms within the aryl ring.

The term “arylalkyl” refers to an aryl group attached to an alkanediyl group where the point of attachment is on the alkanediyl group.

Tauryl/taurate is defined as aminoethyl sulphonyl.

Hetero is defined as an atom other than carbon, including but not exclusive to, nitrogen (N), oxygen (O), or sulfur (S). The heteroatom may be attached in a linear or branched alkyl chain or also may be attached to a non-aromatic or aromatic ring, either as part of the ring, or adjacent to it as a substituent. There may be more than one heteroatom in the alkyl chain or ring.

Unsaturated is defined as a hydrocarbon chain possessing, but not limited, at least one carbon-carbon double bond (C═C). The unsaturated chain may possess one double bond (alkenyl), two double bonds (dienyl), multiple double bonds (polyenyl), and/or a carbon-carbon triple bond (acetylenic). The unsaturated bonds may be adjacent (conjugated) relative to each other or separated by additional carbon atoms in the chain.

A “monomer molecule” is defined by the International Union of Pure and Applied Chemistry (IUPAC) as “A molecule which can undergo polymerization thereby contributing constitutional units to the essential structure of a macromolecule.” A polymer is a macromolecule.

“Isolation” as used in reference to removal of hydrolase from solution either before or after reaction with glycolipids.

“Immobilization” as used herein refers to fixation of hydrolase to a solid support so that it is removed from solution. Example of solid supports include silica, organic frameworks, nickel (affinity-based functionalization) nitrilotriacetic acid agerose, and bacterial spores.

“Treat” or “treating” as used herein refers to a composition, meaning to add or apply a material to the composition.

“About” modifies a particular value by referring to a range of plus or minus 20% or less of the stated value (e.g., plus or minus 15% or less, 10% or less, or 5% or less, or even 1% or less).

“Apply” or “application,” as used herein refers to a composition, meaning to apply or spread the composition onto a human keratinous surface such as the skin or hair.

“Personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, and throat sprays; leave-on skin lotions and creams, shampoos, body washes, body rubs, such as Vicks VapoRub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats. Further non-limiting examples include hand soaps, hand sanitizers, body washes, shower gels, shampoos, body lotions, feminine care products, foot care products, deodorants, pet care products and combinations thereof. Further non-limiting examples include a wipe product suitable for personal care use and household cleaning; a toilet tissue; a towel for hand drying, household drying and household cleaning; a facial tissue; a skin care composition; a first aid or surgical antiseptic; a diaper; a feminine napkin; and combinations thereof.

The term “detergent composition” refers to a composition or formulation designed for cleaning soiled surfaces. Such compositions include but are not limited to, dishwashing compositions, laundry detergent compositions, fabric softening compositions, fabric enhancing compositions, fabric freshening compositions, laundry pre-wash, laundry pretreat, laundry additives, spray products, dry cleaning agent or composition, laundry rinse additive, wash additive, post-rinse fabric treatment, ironing aid, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-cleaning treatment, a post-cleaning treatment, or may be added during the rinse or wash cycle of the cleaning process. The detergent compositions may have a form selected from liquid, powder, single-phase or multi-phase unit dose or pouch form, tablet, gel, paste, bar, or flake. Preferably the composition is for manual washing. The detergent composition of the present invention may be a dishwashing detergent. The composition may be in the form of a liquid. Further non-limiting examples include hard surface cleaners, deodorizers, fabric care compositions, fabric cleaning compositions, manual dish detergents, automatic dish detergents, floor waxes, kitchen cleaners, bathroom cleaners and combinations thereof.

“Cleansing composition” refers to a personal care composition or product intended for use in cleaning a bodily surface such as skin or hair. Some non-limiting examples of cleansing compositions are shampoos, conditioners, conditioning shampoos, shower gels, liquid hand cleansers, facial cleansers, and the like.

“Cosmetic agent” means any substance, as well any component thereof, intended to be rubbed, poured, sprinkled, sprayed, introduced into, or otherwise applied to a mammalian body or any part thereof to provide a cosmetic effect. Cosmetic agents may include substances that are Generally Recognized as Safe (GRAS) by the US Food and Drug Administration and food additives.

“Gel network phase” or “dispersed gel network phase” refers to a lamellar or vesicular solid crystalline phase that includes at least one fatty alcohol, at least one gel network surfactant, and a liquid carrier. The lamellar or vesicular phase can be formed of alternating layers with one layer including the fatty alcohol and the gel network surfactant and the other layer formed of the liquid carrier.

“Solid crystalline” refers to the crystalline structure of the lamellar or vesicular phase at ambient temperatures caused by the phase being below its melt transition temperature. For example, the melt transition temperature of the lamellar or vesicular phase may be about 30° C. or more (i.e., slightly above about room temperature). The melt transition temperature can be measured through differential scanning calorimetry, which is conventional measurement method known to those skilled in the art.

“Suitable for application to human hair” means that the personal care composition or components thereof, are acceptable for use in contact with human hair and the scalp and skin without undue toxicity, incompatibility, instability, allergic response, and the like.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate. “Sulfate-free surfactant” refers to a surfactant that has no sulfate moieties.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

This is not an exhaustive list of sugars, and there are many other sugars with different structures: Glucose is a monosaccharide and is the primary source of energy in living organisms. Its chemical formula is C6H12O6. Fructose is another monosaccharide and is commonly found in fruits and honey. Its chemical formula is also C6H12O6. Galactose is a monosaccharide that is found in milk and dairy products. Its chemical formula is C6H12O6. Sucrose, also known as table sugar, is a disaccharide composed of glucose and fructose. Its chemical formula is C12H22O11. Lactose is a disaccharide found in milk and dairy products. It consists of glucose and galactose. Its chemical formula is C12H22O11. Maltose is a disaccharide formed by the combination of two glucose molecules. It is commonly found in germinating grains. Its chemical formula is C12H22O11. Ribose is a monosaccharide that is an essential component of RNA (ribonucleic acid) and plays a role in energy metabolism. Its chemical formula is C5H10O5. Deoxyribose is a monosaccharide that is a component of DNA (deoxyribonucleic acid). It is similar to ribose but lacks one oxygen atom. Its chemical formula is C5H10O4.

### Rhamnolipid Surfactant

Rhamnose (Rha, Rham), formula illustrated below, is a naturally occurring deoxy sugar. It can be classified as either a methyl-pentose or a 6-deoxy-hexose.

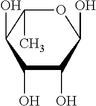

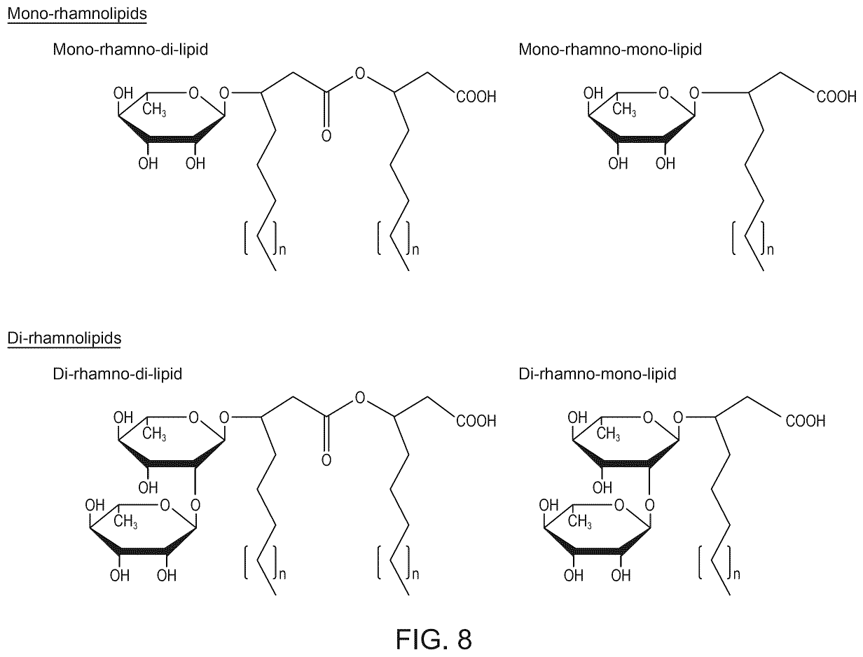

Rhamnolipids are a class of glycolipid that may be used as bacterial surfactants. They have a glycosyl head group, in this case a rhamnose moiety, and an acid fatty tail. There are two main classes of rhamnolipids: mono-rhamnolipids and di-rhamnolipids, which consist of one or two rhamnose groups respectively. And then mono-rhamnolipids and di-rhamnolipids may each have either one or two lipids, creating, for example, the combinations shown in FIG. 8.

The compositions described herein include one or more rhamnolipid biosurfactants. The rhamnolipid surfactants herein may be produced by microorganisms (e.g., Pseudomonas aeruginosa, Pseudomonas putida, Pseudomonas chlororaphis). The rhamnolipid surfactant(s) may provide a cleaning benefit due to their amphiphilic nature, which allows the surfactants to break up, and form micelles around, oil and other contaminants. The “entrapped” contaminant can then be rinsed off more easily with water. A description of various types of rhamnolipids is disclosed in EP2410039. Certain methods of making, extracting, and blending naturally produced rhamnolipids are known in the art.

### Carboxy Ester Hydrolases

In an embodiment of the present invention, commercial rhamnolipids, such as Rheance One, sold by Evonik, or others sold by BioReNuva and Wanhua, may be hydrolyzed by a hydrolase, such as a carboxylic ester hydrolase. The predominantly rhamnodilipids are almost completely hydrolyzed, resulting in predominantly rhamnomonolipids. The carboxylic ester hydrolase may be selected from, but not limited to, the group consisting of esterases, lipases phospholipases, lactonases, tannases, aminoacyl-tRNA hydrolases, cutinases and hydrolases.

Esterases: In the context of the current application, an “esterase” is an enzyme that is capable of catalyzing the splitting of an ester bond into a carboxylic acid and an alcohol functional group.

Lipases: A lipase is an enzyme that catalyzes the hydrolysis of fats or fatty esters into fatty acids and an alcohol functional group. Phospholipases are lipases that hydrolyze phospholipids into fatty acids and other lipophilic compounds.

Lactonases: An enzyme that catalyzes the hydrolysis of a lactone group to open it up into a carboxylic acid and alcohol functional groups.

Cutinase: An enzyme that catalyzes the hydrolysis of a carboxylic ester of a cutin polymer into an alcohol and carboxylic monomer.

Hydrolase: An enzyme that catalyzes the breaking of a bond using water.

The present invention includes a hydrolase (EC 3.1.1.1) that is referred to as eh7 or Est-14, terms that may be used interchangeably.

The present invention also includes variants of enzymes. Variants of enzymes, as used herein, include a sequence resulting when a wild-type protein of the respective protein is modified by, or at, one or more amino acids (for example 1, 2, 5 or 10 amino acids). The invention also includes variants in the form of truncated forms derived from a wild-type enzyme, such as a protein with a truncated N-terminus or a truncated C-terminus. Some enzymes may include an N-terminal signal peptide that is likely removed upon secretion by the cell. The present invention includes variants without the N-terminal signal peptide.

Bioinformatic tools, such as SignalP ver 4.1 (Petersen TN., Brunak S., von Heijne G. and Nielsen H. (2011), Nature Methods, 8:785-786), can be used to predict the existence and length of such signal peptides. The invention also includes variants derived by adding an extra amino acid sequence to a wild-type protein, such as for example, an N-terminal tag, a C-terminal tag or an insertion in the middle of the protein sequence. Non-limiting examples of tags are maltose binding protein (MBP) tag, glutathione S-transferase (GST) tag, thioredoxin (Trx) tag, His-tag, and any other tags known by those skilled in art. Tags can be used to improve solubility and expression levels during fermentation or as a handle for enzyme purification.

It is important that variants of enzymes retain and preferably improve the ability of the wild-type protein to catalyze the conversion of the unsaturated fatty acids. Some performance drop in a given property of variants may of course be tolerated, but the variants should retain and preferably improve suitable properties for the relevant application for which they are intended. Screening of variants of one of the wild-types can be used to identify whether they retain and preferably improve appropriate properties.

The variants may have “conservative” substitutions. Suitable examples of conservative substitution include one conservative substitution in the enzyme, such as a conservative substitution in SEQ ID NO: 11. Other suitable examples include 10 or fewer conservative substitutions in the protein, such as five or fewer. An enzyme of the invention may therefore include 1, 2, 3, 4, 5, 6, 7, 8, 9, 10 or more conservative substitutions.

An enzyme can be produced to contain one or more conservative substitutions by manipulating the nucleotide sequence that encodes that enzyme using, for example, standard procedures such as site-directed mutagenesis or PCR.

Examples of amino acids which may be substituted for an original amino acid in an enzyme and which are regarded as conservative substitutions include: Gly for Ala; Lys for Arg; Gln or His for Asn; Glu for Asp; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

The variants may also have “non-conservative” substitutions, where the resultant amino acid has different properties from the original. Examples of amino acids which may be substituted for an original amino acid in an enzyme and which are regarded as non-conservative substitutions includes: Tyr for Ala; Asp for Gly and Lys for Pro.

A variant includes a “modified enzyme” or a “mutant enzyme” which encompasses proteins having at least one substitution, insertion, and/or deletion of an amino acid. A modified enzyme may have 1, 2, 3, 4, 5, 6, 7, 8, 9 or 10 or more amino acid modifications (selected from substitutions, insertions, deletions and combinations thereof). Enzymes can be modified by a variety of chemical techniques to produce derivatives having essentially the same or preferably improved activity as the unmodified enzymes, and optionally having other desirable properties. For example, carboxylic acid groups of the protein, whether carboxyl-terminal or side chain, may be provided in the form of a salt of a pharmaceutically-acceptable cation or esterified, for example to form a C1-C6 alkyl ester, or converted to an amide, for example of formula CONR1R2 wherein R1 and R2 are each independently H or C1-C6 alkyl, or combined to form a heterocyclic ring, such as a 5- or 6-membered ring. Amino groups of the enzyme, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C1-C20 alkyl or dialkyl amino or further converted to an amide. Hydroxyl groups of the protein side chains may be converted to alkoxy or ester groups, for example C1-C20 alkoxy or C1-C20 alkyl ester, using well-recognized techniques. Phenyl and phenolic rings of the protein side chains may be substituted with one or more halogen atoms, such as F, CI, Br or I, or with C1-C20 alkyl, C1-C20 alkoxy, carboxylic acids and esters thereof, or amides of such carboxylic acids. Methylene groups of the protein side chains can be extended to homologous C2-C4 alkylenes. Thiols can be protected with any one of a number of well-recognized protecting groups, such as acetamide groups. Those skilled in the art will also recognize methods for introducing cyclic structures into the proteins of this disclosure to select and provide conformational constraints to the structure that result in enhanced stability.

Identity percentages were calculated from alignments generated via MUltiple Sequence Comparison by Log-Expectation (MUSCLE) version 5.1 (Edgar, RC (2021), MUSCLE v5 enables improved estimates of phylogenetic tree confidence by ensemble bootstrapping, bioRxiv 2021.06.20) with the Super5 algorithm.

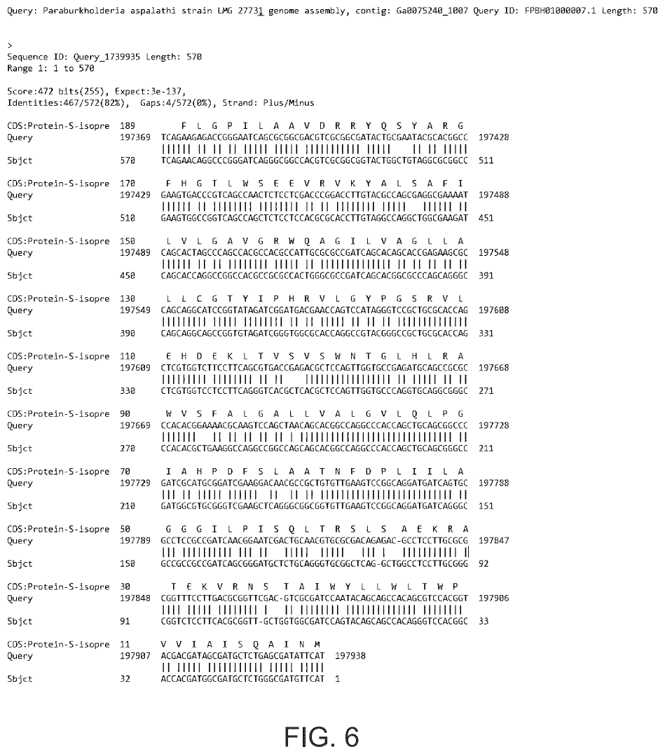

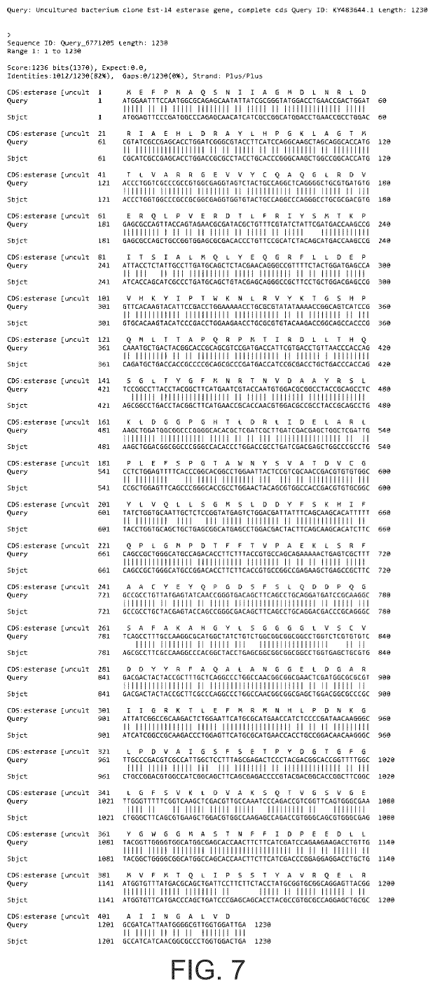

For enzyme sequence comparison the following settings can be used: Alignment algorithm: Needleman and Wunsch, J. Mol. Biol. 1970, 48: 443-453. As a comparison matrix for amino acid similarity the Blosum62 matrix is used (Henikoff S. and Henikoff J. G., PNAS. USA 1992, 89: 10915-10919). The following gap scoring parameters are used: Gap penalty: 12, gap length penalty: 2, no penalty for end gaps. A given sequence is typically compared against the full-length sequence of (SEQ ID NOs: 20 and 4) FIG. 6 and (SEQ ID NOs: 21 and 5) as shown in FIG. 7.

As used herein, “amplify,” “amplified,” or “amplification” refers to any process or protocol for copying a polynucleotide sequence into a larger number of polynucleotide molecules, e.g., by reverse transcription, polymerase chain reaction, and ligase chain reaction.

As used herein, an “antisense sequence” refers to a sequence that specifically hybridizes with a second polynucleotide sequence.

As used herein, “cDNA” refers to a DNA that is complementary or identical to an mRNA, in either single stranded or double stranded form. As used herein, the carbons in fatty acids are numbered with the first carbon as part of the carboxylic acid group, and the second carbon adjacent to the first. The numbers continue so that the highest number carbon is farthest from the carboxylic acid group.

As used herein, “complementary” refers to a polynucleotide that can base pair with a second polynucleotide. For example, a polynucleotide having the sequence 5′-GTCCGA-3′ is complementary to a polynucleotide with the sequence 5′-TCGGAC-3′.

As used herein, a “conservative substitution” refers to the substitution in a polypeptide of an amino acid with a functionally similar amino acid.

As used herein, “encoding” refers to the inherent property of nucleotides to serve as templates for synthesis of other polymers and macromolecules. Unless otherwise specified, a “nucleotide sequence encoding an amino acid sequence” includes all nucleotide sequences that are degenerate versions of each other and that encode the same amino acid sequence.

As used herein, “endogenous” refers to polynucleotides, polypeptides, or other compounds that are expressed naturally or originate within an organism or cell. That is, endogenous polynucleotides, polypeptides, or other compounds are not exogenous.

As used herein, “expression vector” refers to a vector comprising a recombinant polynucleotide comprising expression control sequences operatively linked to a nucleotide sequence to be expressed. For example, suitable expression vectors can be an autonomously replicating or integrated into the chromosome.

As used herein, “exogenous” refers to any polynucleotide or polypeptide that is not naturally expressed in the particular cell or organism where expression is desired. Exogenous polynucleotides, polypeptides, or other compounds are not endogenous.

As used herein, “hybridization” includes any process by which a strand of a nucleic acid joins with a complementary nucleic acid strand through base-pairing. Thus, the term refers to the ability of the complement of the target sequence to bind to a test sequence, or vice-versa.

As used herein, “hybridization conditions” are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm −5° C. (5° C. below the Tm of the probe); “high stringency” at about 5-10° C. below the Tm; “intermediate stringency” at about 10-20° C. below the Tm of the probe; and “low stringency” at about 20-25° C. below the Tm. Alternatively, or in addition, hybridization conditions can be based upon the salt or ionic strength conditions of hybridization and/or one or more stringency washes. For example, 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe.

As used herein, “identical” or percent “identity,” in the context of two or more polynucleotide or polypeptide sequences, refers to two or more sequences that are the same or have a specified percentage of nucleotides or amino acid residues that are the same, when compared and aligned for maximum correspondence, as measured using sequence comparison algorithms or by visual inspection.

As used herein, “long-chain fatty acids” refers to fatty acids with aliphatic tails longer than 14 carbons.

As used herein, “medium-chain fatty acids” refers to fatty acids with aliphatic tails between 6 and 14 carbons. In certain embodiments, the medium-chain fatty acids can have from 11 to 13 carbons.

As used herein, “naturally-occurring” refers to an object that can be found in nature. For example, a polypeptide or polynucleotide sequence that is present in an organism (including viruses) that can be isolated from a source in nature and which has not been intentionally modified by man in the laboratory is naturally-occurring.

As used herein, “operably linked”, when describing the relationship between two DNA regions or two polypeptide regions, simply means that they are functionally related to each other. For example, a promoter is operably linked to a coding sequence if it controls the transcription of the sequence; a ribosome binding site is operably linked to a coding sequence if it is positioned so as to permit translation; and a sequence is operably linked to a peptide if it functions as a signal sequence, such as by participating in the secretion of the mature form of the protein.

As used herein, “polynucleotide” refers to a polymer composed of nucleotides. The polynucleotide may be in the form of a separate fragment or as a component of a larger nucleotide sequence construct, which has been derived from a nucleotide sequence isolated at least once in a quantity or concentration enabling identification, manipulation, and recovery of the sequence and its component nucleotide sequences by standard molecular biology methods, for example, using a cloning vector. When a nucleotide sequence is represented by a DNA sequence (i.e., A, T, G, C), this also includes an RNA sequence (i.e., A, U, G, C) in which “U” replaces “T”

As used herein, “polypeptide” refers to a polymer composed of amino acid residues which may or may not contain modifications such as phosphates and formyl groups.

As used herein, “recombinant expression vector” refers to a DNA construct used to express a polynucleotide that encodes a desired polypeptide and which can include, for example, a transcriptional subunit comprising an assembly of genetic elements having a regulatory role in gene expression, for example, promoters and enhancers, a structural or coding sequence which is transcribed into mRNA and translated into protein, and appropriate transcription and translation initiation and termination sequences. Recombinant expression vectors can be constructed in any suitable manner. The nature of the vector is not critical, and any vector may be used, including plasmid, virus, bacteriophage, and transposon. Possible vectors for use in the present invention include, but are not limited to, chromosomal, nonchromosomal and synthetic DNA sequences, e.g., bacterial plasmids; phage DNA; yeast plasmids; vectors derived from combinations of plasmids and phage DNA, DNA from viruses such as vaccinia, adenovirus, fowl pox, baculovirus, SV40, and pseudorabies.

As used herein, “primer” refers to a polynucleotide that is capable of specifically hybridizing to a designated polynucleotide template and providing a point of initiation for synthesis of a complementary polynucleotide when the polynucleotide primer is placed under conditions in which synthesis is induced.

As used herein, “recombinant polynucleotide” refers to a polynucleotide having sequences that are not naturally joined together. A recombinant polynucleotide may be included in a suitable vector, and the vector can be used to transform a suitable host cell. A host cell that comprises the recombinant polynucleotide is referred to as a “recombinant host cell.” The gene is then expressed in the recombinant host cell to produce, e.g., a “recombinant polypeptide.”

As used herein, “specific hybridization” refers to the binding, duplexing, or hybridizing of a polynucleotide preferentially to a particular nucleotide sequence under stringent conditions.

As used herein, “stringent conditions” refers to conditions under which a probe will hybridize preferentially to its target subsequence, and to a lesser extent to, or not at all to, other sequences.

As used herein, “short-chain fatty acids” refers to fatty acids having aliphatic tails with fewer than 6 carbons.

As used herein, “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using sequence comparison algorithms or by visual inspection. The substantial identity can exist over any suitable region of the sequences, such as, for example, a region that is at least about 50 residues in length, a region that is at least about 100 residues, or a region that is at least about 150 residues. In certain embodiments, the sequences are substantially identical over the entire length of either or both comparison biopolymers.

The compositions resulting from the inventive method may comprise rhamnolipids in which at least about 25% by weight of all the rhamnolipids are monolipids. In some embodiments, the weight % of all rhamnolipids in a composition that are monolipids may be at least about 15%, at least about 20%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or at least about 100%. In some embodiments, the weight % of all rhamnolipids in a composition that are monolipids may be from about 25%, about 30%, about 35%, about 40%, about 45%, about 50%, about 55%, about 60%, about 65%, about 70%, about 75%, about 80%, about 85%, about 90%, about 95%, to about 40%, about 45%, about 50%, about 55%, about 60%, about 65%, about 70%, about 75%, about 80%, about 85%, about 90%, about 95%, about 96%, about 97%, about 98%, about 99%, or about 100%, with any combination of these percentages herein.

In some embodiments, a composition resulting from the inventive method may comprise rhamnolipids in which monorhamnomonolipids comprise at least about 20%, about 30%, about 40%, about 50%, about 60%, about 70%, about 80%, about 90%, about 95%, about 99%, or about 100% by weight of all the rhamnolipids, or in some embodiments from about 20% to about 100%, or about 25% to about 95%, or any combination therebetween, by weight of all rhamnolipids. In other embodiments, the composition may comprise rhamnolipids in which a dirhamnomonolipid comprises at least about 20%, about 30%, about 40%, about 50%, about 60%, about 70%, about 80%, about 90%, about 95%, about 99%, or about 100% by weight of all the rhamnolipids, or in some embodiments from about 20% to about 100%, or about 25% to about 95%, or any combination therebetween, by weight of all rhamnolipids.

In some embodiments, the rhamnodilipids may be at most about 10%, at most about 15%, at most about 20%, at most about 25% by weight of all rhamnolipids of a final composition. In some embodiments, monorhamnodilipids may be at most about 10%, at most about 15%, at most about 20%, at most about 25% by weight of all rhamnolipids. In some embodiments, dirhamnodilipids may be at most about 10%, at most about 15%, at most about 20%, at most about 25% by weight of all rhamnolipids. In one embodiment, the di-rhamno-di-lipids are at most about 25 wt % of all rhamnolipid compositions present in the composition.

### Antimicrobial or Preservative

The compositions that result from the inventive methods herein may be used as part of a personal care composition or a detergent composition. In some embodiments the compositions may have effect as a surfactant and/or as an antimicrobial and/or as a preservative. In some embodiments, the present invention may produce an antimicrobial composition. In some embodiments, the present invention may be a personal care product or composition comprising the composition made by an inventive method. In some embodiments, the present invention may be a method of reducing or killing bacteria by using or comprising the composition made through the inventive methods. In some embodiments, such methods may be used to reduce microbial population of formulated products, biological tissue or inanimate materials. In some embodiments, the formulated products, biological tissue, or inanimate materials may contact the composition for sufficient time to provide substantial microbial reduction. The amount of composition in a personal care composition, a detergent composition, a preservative composition, or an antimicrobial composition may be from about 0.1 to about 25% by weight of the composition, from about 0.2 to about 15% by weight of the composition, or from 0.5 to about 12% of the composition.

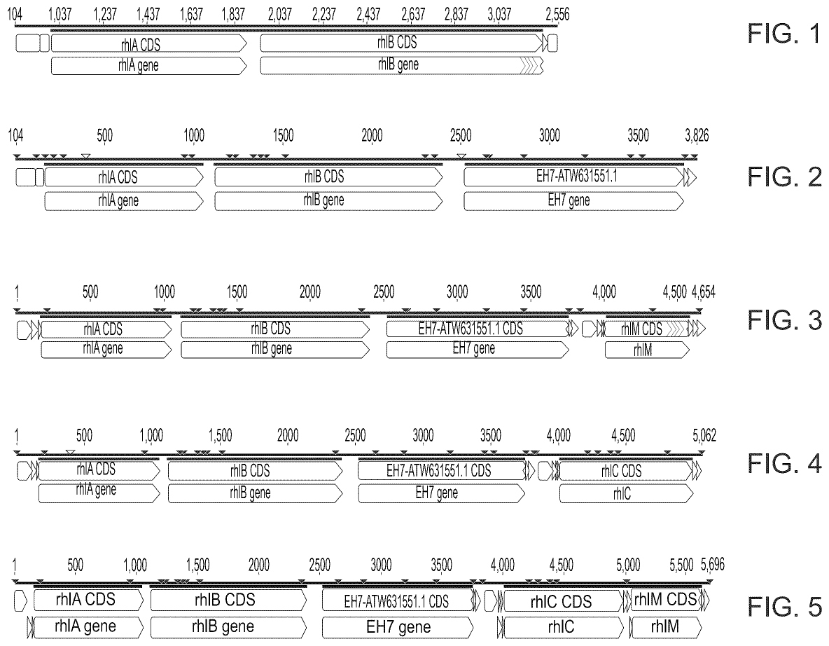

FIG. 1 is a map for pPG1 insert which includes an initial terminator to prevent plasmid readthrough, synthetic promoter (P_{ProSyn8}), ribosomal binding site (RBS), followed by rhlA (SEQ ID NO: 1) and rhlB (SEQ ID NO: 2) genes, and subsequent terminator to end transcription in the 3′UTR (untranslated region).

FIG. 2 is a map for pPG2 insert which includes an initial terminator to prevent plasmid readthrough, synthetic promoter (P_{ProSyn8}), ribosomal binding site (RBS), followed by rhlA (SEQ ID NO: 1), rhlB (SEQ ID NO: 2), eh7 (SEQ ID NO: 5) genes, and subsequent terminator to end transcription in the 3′UTR (untranslated region).

FIG. 3 is a map for pPG6 insert which includes an initial terminator to prevent plasmid readthrough, synthetic promoter (P_{ProSyn8}), ribosomal binding site (RBS), followed by rhlA (SEQ ID NO: 1), rhlB (SEQ ID NO: 2), eh7 (SEQ ID NO: 5) genes, and subsequent terminator to end transcription in the 3′UTR. A second transcript is downstream with initial terminator, synthetic promoter (P_{ProSyn8}), RBS, and rhlM (SEQ ID NO: 4) followed by a subsequent terminator in the 3′UTR(untranslated region).

FIG. 4 is a map for pPG7 insert which includes an initial terminator to prevent plasmid readthrough, synthetic promoter (P_{ProSyn8}), ribosomal binding site (RBS), followed by rhlA (SEQ ID NO: 1), rhlB (SEQ ID NO: 2), eh7 (SEQ ID NO: 5) genes, and subsequent terminator to end transcription in the 3′UTR. A second transcript is downstream with initial terminator, synthetic promoter (P_{ProSyn8}), RBS, and rhlC (SEQ ID NO: 3) followed by a subsequent terminator in the 3′UTR (untranslated region).

FIG. 5 is a map for pPG8 insert which includes an initial terminator to prevent plasmid readthrough, synthetic promoter (P_{ProSyn8}), ribosomal binding site (RBS), followed by rhlA (SEQ ID NO: 1), rhlB (SEQ ID NO: 2), eh7 (SEQ ID NO: 5) genes, and subsequent terminator to end transcription in the 3′UTR. A second transcript is downstream with initial terminator, synthetic promoter (P_{ProSyn8}), RBS, and rhlC (SEQ ID NO: 3) and rhlM (SEQ ID NO: 4) followed by a subsequent terminator in the 3′UTR (untranslated region).

### Example A

### The Preparation of Hydrolase SEQ ID NO: 12

The hydrolase (SEQ ID NO: 11) is a carboxylesterase enzyme (EC 3.1.1.1) that converts esterified di-fatty acids (e.g., 3-(3-hydroxyalkanoyloxy)alkanoate, HAA) into corresponding monomeric fatty acids. A codon optimized gene (SEQ ID NO: 6) encoding for an uncultured bacterium clone Est-14 esterase, including an N-terminal amino acid sequence containing a His-tag and a Thrombin protease cleavage site (SEQ ID NO: 12), was designed and synthesized. After gene synthesis, the protein was expressed and purified. In brief, the complete synthetic gene sequence was subcloned into a pET15b vector (GenScript Biotech, Piscataway, NJ, USA) for heterologous expression. Escherichia coli BL21 Star (DE3) cells (Invitrogen, Waltham, MA, USA) were transformed with the recombinant plasmid, grown overnight on a Luria broth agarose plate containing 100 μg/mL carbenicillin (Teknova, Hollister, CA, USA) at 37° C. A single colony was inoculated into Luria broth (LB, Fisher Scientific, Waltham, MA, USA) containing 100 μg/mL carbenicillin (Teknova, Hollister, CA, USA). Magic E. coli expression medium (Invitrogen, Waltham, MA, USA) was inoculated with culture grown in LB at a 1:50 dilution. Cultures were incubated at 31° C. for 24 hr at 175 rcf and auto-induction was achieved through the components of magic medium to induce protein expression. Cells were harvested by centrifugation at 4000 rcf for 30 min and the cell pellets were lysed by adding 5 mL of B-PER bacterial protein extraction reagent (Thermo Scientific, Waltham, MA, USA) supplemented with DNAse I, Lipase and HALT protease inhibitors (Thermo Scientific, Waltham, MA, USA) per 1 gram of cell pellet and allowing to lyse at room temperature for 90 minutes, rocking. After centrifugation at 14,000 rcf, 4° C., the supernatant was collected and the protein was purified by one-step purification using a nickel affinity column (Histrap HP, Cytiva, Marlborough, MA, USA) wherein protein was eluted and collected using 5 column volumes of 0.5 M imidazole (Thermo Scientific, Waltham, MA, USA), 10 mM HEPES, pH 8.0. Resulting elutant was then dialyzed in a 10,000 Da molecular weight cutoff dialysis cassette (Pierce, Waltham, MA, USA) against a buffer containing 10 mM HEPES, at pH 8.0.

### Example B

### Preparation of Rhamnomonolipids by hydrolase

Hydrolase and rhamnodilipid (Rheance One in this example, though other fermented mixtures or chemically synthesized dilipids may alternatively be used, Evonik Industries AG, Essen, Germany) is mixed with 0.5 M HEPES (Sigma-Aldrich, Saint Louis, MO, USA). The mixture was incubated at 37° C. under 175 rcf agitation and allowed 2-24 hours for the reaction to reach completion. Reaction completion was monitored via thin layer chromatography (TLC) on silica gel 60 (Supelco, Bellefonte, PA, USA) by spotting the sample then using 50:15:2 Chloroform/Methanol/Acetic Acid (Sigma-Aldrich, Saint Louis, MO, USA) as a mobile phase. Once completed, the plate was allowed to dry and derivatized using Ceric Ammonium Molybdate (CAM; TCI America, Portland, OR, USA) and/or orcinol (Sigma-Aldrich, Saint Louis, MO, USA) as a stain. Reaction was considered complete whenever no starting material was visible by TLC. Final product was then analyzed via UPLC-CAD (ultra high-performance liquid chromatography—charged aerosol detector) to determine quantitative extent of hydrolysis. Table 1.3a, on a sample from Example B, shows the retention time from 2 to 12 minutes of Rheance One before and after hydrolysis. A rhamnose group is represented as (Rha) and a lipid abbreviated as Cn or Cn:m (where n=number of carbon atoms; m=number of unsaturation). ND=not detected. % Difference of molecule post hydrolysis=(AUC post hydrolysis)/(AUC before hydrolysis)*100.

The data shows that about 79.4% by weight of the inventive composition is rhamnomonolipids. The retention time of the readings captures all rhamnolipids in the composition, though there may be additional materials, such as salts and unhydrolyzed starting components. As can be seen in Table 1.3b, the inventive composition comprises a much higher amount of rhamnomonolipid material.

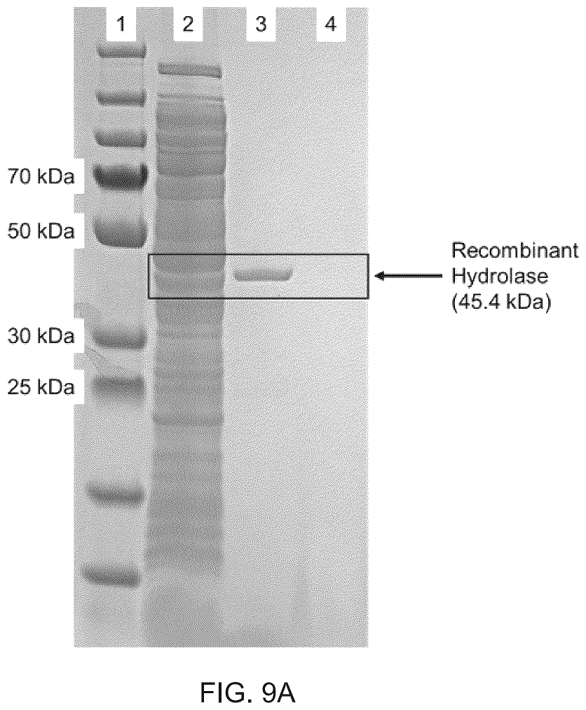

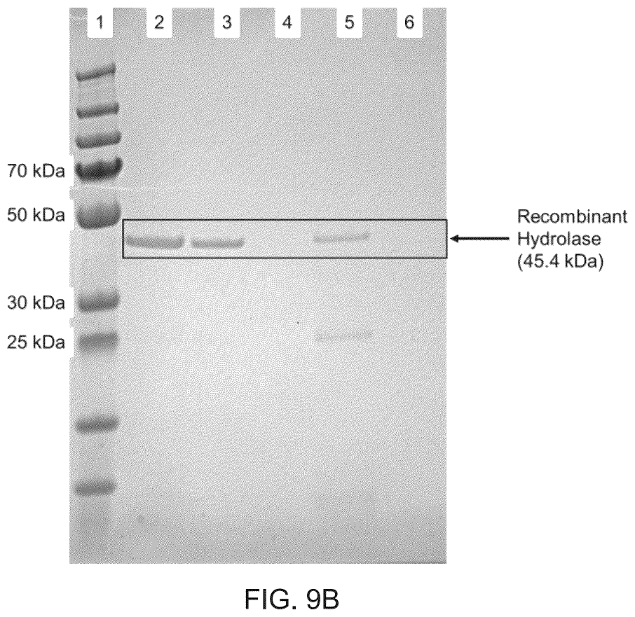

Recombinant hydrolase can further be isolated from reactive solution by directly applying the reaction solution to a nickel affinity column (Cytiva, Marlborough, MA, USA) and collecting the resultant flow through. Enzyme isolation was verified via SDS-PAGE wherein resultant flow through was denatured at 100° C. for 10 minutes in the presence of reducing agent (Invitrogen, Waltham, MA, USA). Following denaturation, samples and molecular weight marker (PageRuler Plus; Invitrogen, Waltham, MA, USA) were loaded onto 4-12% Bis-Tris polyacrylamide gels (Invitrogen, Waltham, MA, USA and run at continuous voltage of 150 V until completion. The polyacrylamide gel was then rinsed with deionized water and stained with Coomassie (Imperial protein stain; Thermo Scientific, Waltham, MA, USA) to observe protein bands. The absence of a band indicated that enzyme had been isolated/removed within the lower limit of detection of the stain (FIG. 9A). The isolated enzyme can then be eluted from the nickel affinity column using 25 mL of 0.5 M imidazole in 10 mM HEPES pH 8.0 and then dialyzed using a cassette with a 10,000 Da molecular weight cut off (Pierce, Waltham, MA, USA) against a buffer containing 10 mM HEPES, pH 8.0. Following dialysis, enzyme could then be used in a subsequent reaction as described above (FIG. 9B).

TABLE 1.3a
Table of Rhamnolipid species before and after hydrolase reaction
Peak List
Retention Time (RT) from 2 to 12 minutes

| | | | | Analyte |
|---|---|---|---|---|
| RT | Area | Wt. % | Analyte | (cont.) |
| 0.52 | 0.108 | 0.21 | | |
| 0.56 | 0.339 | 0.67 | | |
| 0.81 | 0.045 | 0.09 | | |
| 2.67 | 0.025 | 0.05 | | |
| 2.69 | 0.013 | 0.03 | | |
| 2.76 | 0.013 | 0.03 | | |
| 2.8 | 0.017 | 0.03 | | |
| 2.9 | 0.227 | 0.45 | | |
| 3.05 | 0.293 | 0.58 | RhaRha-C8 | |
| 3.55 | 0.017 | 0.03 | | |
| 3.58 | 0.021 | 0.04 | | |
| 3.61 | 0.018 | 0.04 | | |
| 3.68 | 0.005 | 0.01 | | |
| 3.7 | 0.014 | 0.03 | | |
| 3.82 | 0.016 | 0.03 | | |
| 3.95 | 0.064 | 0.13 | | |
| 4.1 | 28.655 | 56.54 | RhaRha-C10 | |
| 4.2 | 11.097 | 21.89 | Rha-C10 | |
| 4.32 | 0.016 | 0.03 | | |
| 4.33 | 0.007 | 0.01 | | |
| 4.39 | 0.078 | 0.15 | | |
| 4.45 | 1.195 | 2.36 | C10 | |
| 4.52 | 0.059 | 0.12 | | |
| 4.56 | 0.058 | 0.11 | | |
| 4.63 | 0.016 | 0.03 | | |
| 4.7 | 0.072 | 0.14 | | |
| 4.8 | 0.01 | 0.02 | | |
| 4.9 | 0.045 | 0.09 | | |
| 5.02 | 1.17 | 2.31 | Rha-C12 | |
| 5.09 | 0.02 | 0.04 | | |
| 5.16 | 0.175 | 0.35 | | |
| 5.22 | 0.37 | 0.73 | | |
| 5.28 | 0.247 | 0.49 | | |
| 5.34 | 0.041 | 0.08 | | |
| 5.45 | 0.022 | 0.04 | | |
| 5.51 | 0.057 | 0.11 | | |
| 5.67 | 0.755 | 1.49 | RhaRha-C10C8 | |
| 5.73 | 0.024 | 0.05 | | RhaRha-C8C10 |
| 5.82 | 0.84 | 1.66 | | |
| 5.91 | 0.012 | 0.02 | | |
| 5.96 | 0.013 | 0.03 | | |
| 6.05 | 0.039 | 0.08 | | |
| 6.15 | 0.153 | 0.30 | | |
| 6.33 | 0.235 | 0.46 | | |
| 6.59 | 0.026 | 0.05 | | |
| 6.68 | 0.137 | 0.270302 | | |
| 6.76 | 0.055 | 0.11 | | |
| 6.92 | 0.032 | 0.06 | | |
| 6.96 | 0.019 | 0.04 | | |
| 7.27 | 0.139 | 0.27 | RhaRha-C10C10 | |
| 7.58 | 0.034 | 0.07 | | |
| 7.89 | 0.031 | 0.06 | C10C10 | |
| 8.15 | 0.011 | 0.02 | | |
| 8.17 | 0.013 | 0.03 | | |
| 8.24 | 0.02 | 0.04 | | |
| 8.68 | 0.022 | 0.04 | | |
| 9.24 | 0.086 | 0.17 | | |
| 9.49 | 1.903 | 3.75 | | |
| 9.77 | 0.077 | 0.15 | | |
| 9.92 | 0.038 | 0.07 | | |
| 10.08 | 0.358 | 0.71 | | |
| 10.28 | 0.364 | 0.72 | | |
| 10.66 | 0.21 | 0.41 | | |
| 10.75 | 0.119 | 0.23 | | |
| 10.95 | 0.118 | 0.23 | | |
| 11.68 | 0.071 | 0.14 | | |
| 11.95 | 0.05 | 0.10 | | |

TABLE 1.3b

| | Before inventive method: | After inventive method: |
|---|---|---|
| | Pre-hydrolysis (wt. %) | Post-hydrolysis material of |
| | (Rheance One): | Example B (wt. %): |
| Rhamnomonolipids | 3.6 | 79.4 |
| Rhamnodilipids | 94 | 7.6 |

FIG. 9A is a SDS-PAGE of isolated recombinant hydrolase on 4-12% Bis-Tris polyacrylamide gel run in MOPS running buffer (Invitrogen, Waltham, MA, USA) showing expression, isolation and removal from reaction solution. Lane 1: PageRuler molecular weight marker; Lane 2: Clarified extract of expressed recombinant hydrolase; Lane 3: Isolated recombinant hydrolase using nickel affinity chromatography; Lane 4: Reactant solution flow through.

FIG. 9B is a SDS-PAGE of reused, recombinant hydrolase on 4-12% Bis-Tris polyacrylamide gel run in MOPS running buffer showing expression and presence in reaction solution. Lane 1: PageRuler molecular weight marker; Lane 2: purified recombinant hydrolase; Lane 3: First reaction solution containing recombinant hydrolase; Lane 4: first reaction solution control lacking recombinant hydrolase; Lane 5: second reaction solution containing recombinant hydrolase; Lane 6: second reaction solution lacking recombinant hydrolase.

TABLE 1.4
Reused enzyme analyte quantification

| | RhaRha- | RhaRha-C10 | C10 | Product/Starting |
|---|---|---|---|---|
| Condition: | C10C10 AUC: | AUC: | AUC: | Material: |
| 0.1% Rheance One alone | 561000000 | 28875 | 5116724 | 0.01 |
| 0.1% Rheance One + hydrolase | 858993 | 1122681 | 211000000 | 246.94 |
| first round | | | | |
| 0.1% Rheance One − hydrolase | 844000000 | 62344 | 10047799 | 0.01 |
| first round | | | | |
| 0.1% Rheance One + hydrolase | 5678678 | 554000000 | 589000000 | 201.28 |
| second round | | | | |
| 0.1% Rheance One − hydrolase | 1420000000 | 57604364 | 56087308 | 0.08 |
| second round | | | | |

### Example C

### Enzyme Isolation or Immobilization

Recombinant hydrolase (SEQ ID NO:12) was first applied to affinity resin (e.g., Nickel affinity resin; HisTrap HP) to isolate or immobilize the enzyme. Then, a 1% w/v solution of rhamnodilipids (e.g., Rheance One) buffered by 0.5 M HEPES to pH 8.0 was flowed through the resin, continuously at 37° C. for 16 hr. To determine the extent of hydrolysis, the column flow through was collected and analyzed through aforementioned methods to determine rate of hydrolysis.

TABLE 1.4
Immobilized enzyme analyte quantification

| | RhaRha-C10C10 | RhaRha-C10 | C10 | Ratio Product/Starting |
|---|---|---|---|---|
| Condition: | Area | Area | Area | Material |
| T = 0 hr | 1810000000 | 1732667 | 143000000 | 0.08 |
| T = 16 hr (No | 1440000000 | 405000000 | 575000000 | 0.68 |
| Ez) | | | | |
| T = 16 hr (+Ez) | 8504836 | 304000000 | 372000000 | 79.48 |

### Example D

### Preparation of Electrocompetent P. putida

P. putida strain KT2440 was made electrocompetent through procedures common in the art. Electrocompetent P. putida was transformed with expression vectors with varying inserts (FIGS. 1-5) and selected via antibiotic resistance (e.g., 50 μg/mL Kanamycin; VWR International, Radnor, PA, USA) with the following electroporation specifications: ECM 630 (BTX) 1.7 kV, 200 Ω, 25 ΩF, time constant 4-5 ms.

### Example E

P. putida KT2440 Expression Cassette

Expression cassettes are shown in FIGS. 1-5.

### Example F

### Preparation of Mono-Rhamnomonolipids by Fermentation

Constructs shown in FIG. 1 pPG1-RhlAB (SEQ ID NO: 1 and SEQ ID NO: 2) and FIG. 2 pPG2-RhlAB-EH7 (SEQ ID NOs: 1, 2 and 5) were expressed via fermentation described in test methods below. Rhamnolipids produced were analyzed as LC/MS and UPLC-CAD as shown in Example B.

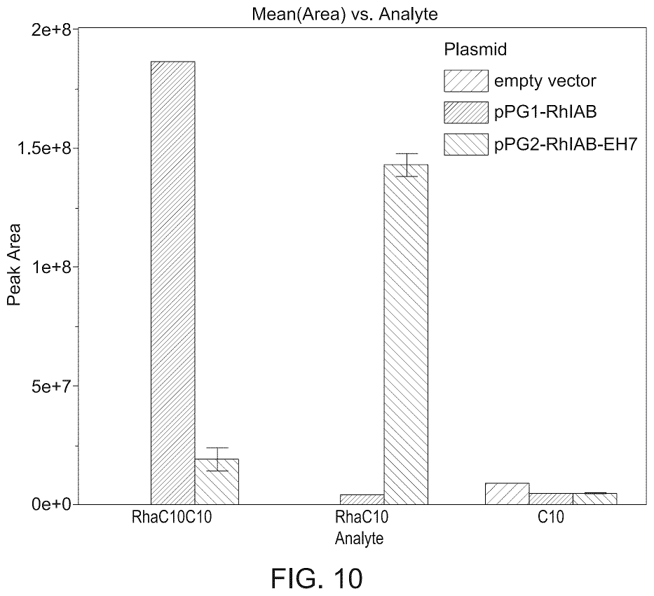

As shown in FIG. 10, rhamnomonolipids were produced when all three genes (SEQ ID NOs: 1, 2 and 5) were expressed.

### Example G

### Preparation of Di-Rhamno-Monolipids by Fermentation

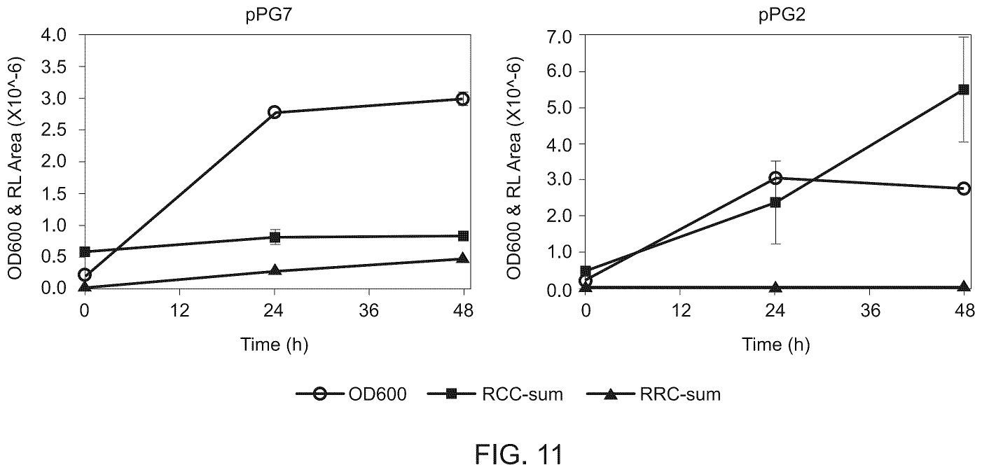

Constructs shown in FIG. 4 pPG7-RhlAB-EH7-RhlC (SEQ ID NOs: 1, 2, 5, and 3) and FIG. 2 pPG2-RhlAB-EH7 (SEQ ID NOs: 1, 2, and 5) were expressed in Pseudomonas putida KT2440 (ATCC 47054) (American Type Culture Collection, Manassas, VA, USA); strains were grown overnight in Luria Broth (Thermo Fisher Scientific, Waltham, MA, USA) plus 10 g/L glucose, and 50 g/mL kanamycin (Thermo Fisher Scientific, Waltham, MA, USA). Twenty-five mL of the described medium was added to 250 mL baffled flasks. Pseudomonas putida KT2440 (ATCC 47054) (American Type Culture Collection, Manassas, VA, USA) expressing either plasmid pPG7 or pPG2 was grown overnight in Luria Broth from a glycerol stock at 30° C. and 225 rpm, then inoculated to a starting OD of 0.2. Conversion was allowed to proceed for 48 hours, 30° C. and 225 rpm, with samples for analytics taken every 24 hours. Optical density was measured on the SpectraMax iD5 (Molecular Devices, San Jose, CA, USA). Samples were analyzed for the presence of rhamnolipid by mass spectrometry. The data in FIG. 11 shows the sum of the detected areas under the peak of all mono-rhamno-di-lipid congeners of various chain lengths (“RCC-sum”) and the sum of all di-rhamno-mono-lipid congeners (“RRC-sum”) of various chain lengths.

As shown in FIG. 11, Pseudomonas putida expressing RhlC was able to accumulate di-rhamno-mono-lipids (RRC, triangles), while the strain harboring plasmid pPG2, without RhlC, was not.

### Preparation of Methylated Rhamnomonolipids by Fermentation

The construct shown in FIG. 5 (SEQ ID NOs: 1, 2, 5, 3 and 4) was expressed via fermentation described in test methods below. Rhamnolipids produced were analyzed as LC/MS and UPLC-CAD as shown in Example B.

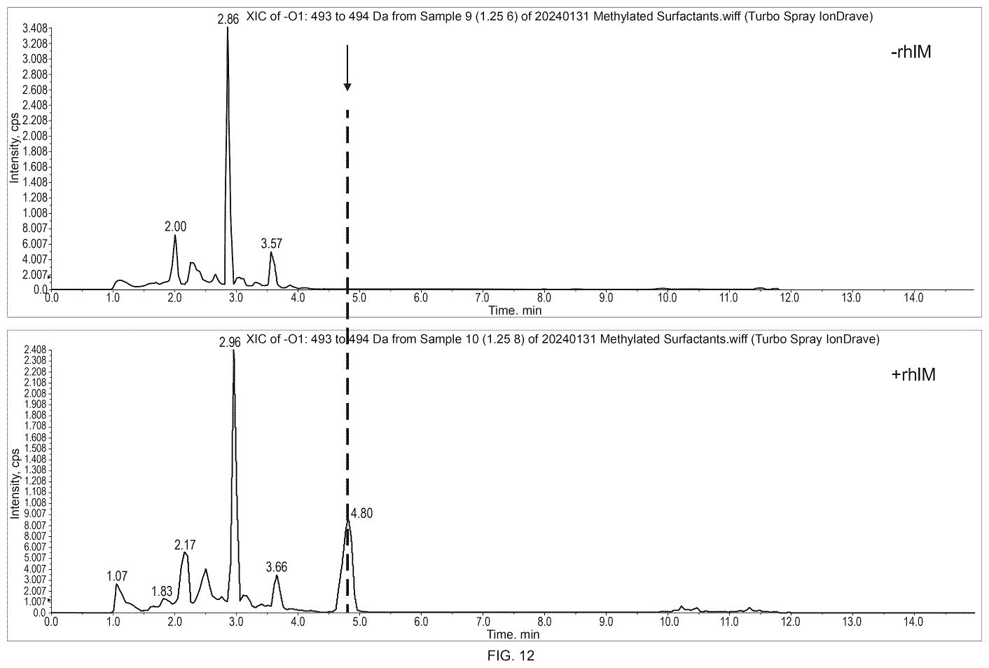

As shown in FIG. 12, P. putida expressing rhlM (SEQ ID NO: 4) produced methylated Rha-C10, RhaRha-C10, and Rha-C10C10 (for example) where these (and other) congeners were not observed in the absence of rhlM.

### Example H

### Preparation of Monorhamno-Monolipid from Complex Feedstocks

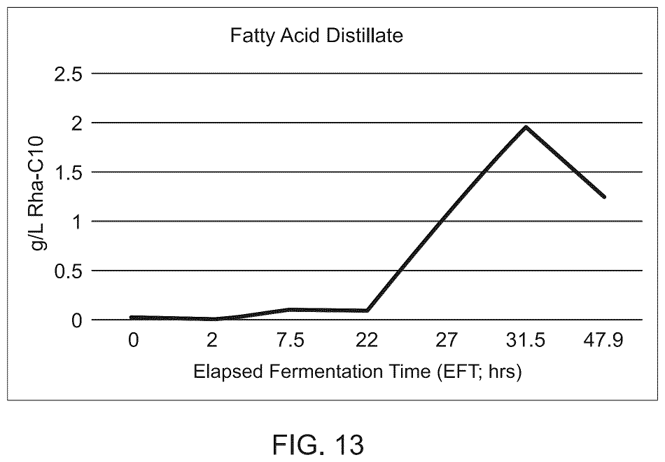

FIG. 13 provides an example of mono-rhamno-mono-lipid production using fatty acid distillate.

As shown in FIG. 13, Pseudomonas putida expressing the construct shown in FIG. 2 pPG2-RhlAB-EH7 (SEQ ID NOs: 1, 2, and 5) was able to utilize fatty acid distillate as a sole carbon source for mono-rhamno-mono-lipid production. Cells were grown at 30° C., without pH control, cascade was set to first increase agitation then sparge to maintain at least 60% dissolved oxygen in minimal media (BSM: 3.4 g/L KH₂PO₄ (Sigma-Aldrich, St. Louis, MO, USA), 4.4 g/L K₂HPO₄ (Sigma-Aldrich, St. Louis, MO, USA), 15.0 g/L NaNO₃ (VWR International, Radnor, PA, USA), 1.1 g/L KCl (Thermo Fisher Scientific, Waltham, MA, USA), 1.1 g/L NaCl (Sigma-Aldrich, St. Louis, MO, USA), 0.5 g/L Yeast Extract (Becton Dickinson, Franklin Lakes, NJ, USA), 0.224 g/L MgSO₄ (VWR), 0.00145 g/L ZnSO₄×7H₂O (Sigma-Aldrich, St. Louis, MO, USA), 0.00125 g/L CuSO₄×5H₂O (Sigma-Aldrich, St. Louis, MO, USA), 0.0084 g/L MnSO₄×H₂O (Spectrum Chemical Mfg. Corp, New Brunswick, NJ, USA), 0.0012 g/L CaCl₂×4H₂O (Sigma-Aldrich, St. Louis, MO, USA), and 0.00028 g/L FeSO₄ (Spectrum Chemical Mfg. Corp, New Brunswick, NJ, USA) and 50 g/mL kanamycin (VWR International, Radnor, PA, USA); Polyglycol (Dow P 2000) (Dow Inc., Midland, MI, USA) was used as the antifoaming agent.

### Example I

### Assessing Foaming Properties of Formulated Rhamnolipids

Table 1.4 provides three examples of shampoo formulations comparing foaming performance of Rheance One, Inventive Example B, and a control containing Rheance One, additional buffer, but no hydrolase (Labeled “No Enzyme Control” SEQ ID NO: 12).

The compositions in Table 1 are made by adding deionized (DI) water to a mixing vessel and heating to 75° C. while stirring. Sodium cocoyl isethionate, if present, is added to the mixing vessel and stirred until it is fully dissolved (i.e., no visible particles remaining and batch is clear). The remaining ingredients, except for polyquaternium-10, are then added to the mixing vessel and mixed for at least 10 minutes. The batch is cooled to 35° C. or less. In a separate container, the polyquaternium-10 is mixed with water at a 1:20 ratio (polyquat: water) to form a slurry, which is then added to the cooled composition in the mixing vessel and mixed for 10 minutes. The pH of the composition is adjusted with sodium hydroxide, and deionized water is added to bring the final volume to 100%. The mixture is mixed until homogeneous (about 10 minutes).

### Blender Lather Volume

This method can be used to evaluate a foam property that consumers associate with the quality of a shampoo product. This method is described in detail in Klein, Ken, “Evaluating Shampoo Foam,” Cosmetic & Toiletries, Vol. 119, No. 10, p 32-35, 2004.

One (1) gram of the shampoo product is added to 39 grams of deionized water at room temperature. This solution is carefully poured into the single-speed Magic Bullet MB1001 blender to minimize the introduction of bubbles and agitated for 10 seconds. The foam is then poured into a graduated cylinder, and the volume of the foam is measured and recorded.

TABLE 1.4

| | Rheance | Inventive | No Enzyme |
|---|---|---|---|
| Ingredients | One | Example B | Control |
| Sodium cocoyl | — | — | — |
| isethionate | | | |
| Sodium lauroyl | — | — | — |
| sarcosinate | | | |
| Alkyl amidopropyl | 9.8 | 9.8 | 9.8 |
| betaine | | | |
| Rheance One | 8.5 | — | — |
| Inventive Example B | — | 8.5 | — |
| No Enzyme Control - | — | — | 8.5 |
| containing Rheance One | | | |
| Sodium benzoate | 0.8 | 0.8 | 0.8 |
| Sodium salicylate | 0.5 | 0.5 | 0.5 |
| Tetrasodium EDTA | 0.2 | 0.2 | 0.2 |
| Polyquaternium-10 | 0.3 | 0.3 | 0.3 |
| Buffer | To pH 6.8 | To pH 6.8 | To pH 6.8 |
| | to 7.2 | to 7.2 | to 7.2 |
| DI Water | qs | qs | qs |

Rheance One brand glycolipid surfactant from Evonik (~50% active), Evonik Industries AG, Essen, Germany.

### Inventive Example B, (100% Active)

No Enzyme control, containing Rheance One, additional buffer, no hydrolase (˜50% active).

TABLE 1.5

| | Rheance | Inventive | No Enzyme |
|---|---|---|---|
| | One | Example B | control |
| Lather volume (mL) | 85 | 145 | 100 |

As summarized in Table 1.5, the inventive composition made by the inventive method exhibited better lather volume than formulated Rheance One or No enzyme control containing Rheance One subjected to the same conditions as the hydrolysate without enzyme.

### Absorbance Scan for Colorimetric Differences

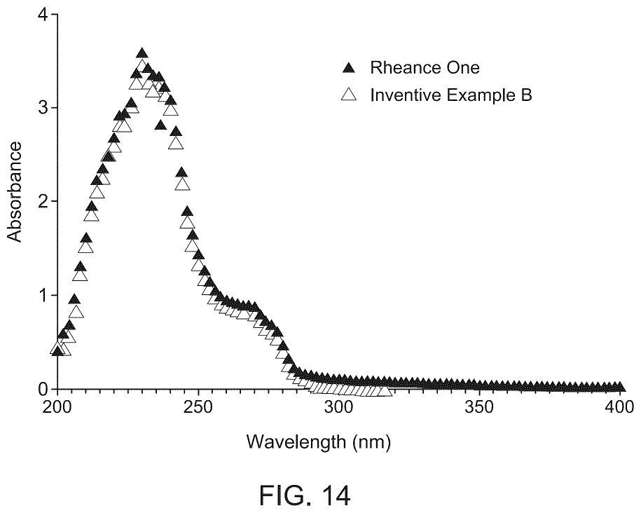

Rheance One and Inventive Example B were fully dissolved in water and any insoluble particulates were removed by passing the sample through a 0.45 μM filter. The samples were placed in quartz cuvettes and then into a spectrophotometer. An absorbance scan was performed from 200-1100 nm with 2 nm increments. Resulting spectra of absorbance as a function of wavelength were then overlaid to determine differences and are shown in FIG. 14.

These data suggest that Rheance One and Inventive Example B have similar spectral signatures. Note that 400-1100 nm was not shown as there was little signal in this range.

In other words, this shows that glycomonolipids made by the inventive method do not exhibit detectable discoloration from the starting glycodilipids and that rhamnomonolipids made by the inventive method do not exhibit detectable discoloration from the starting rhamnodilipids.

### Test Methods

### Analysis by UPLC-CAD

Chromatography consisted of an ACQUITY BEH C18 Vanguard precolumn (130 Å, 1.7 μm, 2.1 mm×5 mm, Waters, Milford, MA, USA) followed by an ACQUITY UPLC BEH C18 Column (130 Å, 1.7 μm, 2.1 mm×100 mm, Waters, Milford, MA, USA). The LC gradient was carried out on Vanquish UPLC-CAD system (Thermo Fisher Scientific, Waltham, MA, USA) from 5 to 95% B (Acetonitrile) over 10.5 min, for a total run time of 15 min with inverse gradient. Data in Table 1.3 represents results from the retention time of 2 to 12 minutes. Mobile Phase A was 4 mM Ammonium Acetate in Water, and the flow rate was set to 0.4 mL/min. The column temperature was kept at 45° C. in the column compartment. All data processing was performed using the Xcalibur Software.

The peak identity for rhamnolipids is confirmed by LC-MS/MS (liquid chromatography (LC) tandem mass spectrometry (MS).

Eluted rhamnolipids from the UPLC column were analyzed by tandem mass spectrometry on a Triple Quad 6500+ system (AB Sciex, Framingham, MA, USA). The instrument was operated in multiple reaction monitoring (MRM) and negative electrospray ionization (ESI) mode.

All data processing was performed using the Skyline Software, Version 23.1.0.268 (MacCoss Lab, University of Washington, Seattle, WA, USA). The transition results were exported to CSV file for further analysis. For relative quantitation purpose, carbon-13 stable isotope labeled Rhamnolipids was prepared by fermentation using D-glucose (U-13C6) as the sole carbon source. The list of rhamnolipids and corresponding transitions is included.

### Fermentation of Microbial Cultures

Suitable conditions consisting of a fermentation broth having a concentration of rhamnolipids were comprised of: a bacterial seed culture, preferably a strain of Pseudomonas putida bacteria; preparing a fermentation medium in a suitable fermentation vessel equipped to agitate and aerate a fermentation broth, the fermentation medium comprising a carbon source selected from the group selected from fatty acid distillate, used soybean oil, soybean oil soapstock, orange peels, distillery waste, wheat straw, sweet water, or sugarcane begasse oil, soybean oil, rapeseed oil, cocoa butter, olive oil, rice bran oil, palm oil, animal fat, glycerol, fatty acids, used cooking oil, waste oil, waste grease, glucose, fructose, sucrose, lactose, maltose, corn syrup, corn molasses, soy molasses, carbohydrates, materials containing carbohydrates, glycerides, fatty acids, glycerol, and combinations thereof, a nitrogen source and a non-nitrogen source and combinations therein. Aion and aeration of the fermentation broth was performed at a temperature of from 15° C. to 40° C.; more preferably 30° C.

### Examples/Combinations

A. A method of making glycomonolipids; said method comprising the following steps:
   a. reacting glycodilipids with a hydrolase to form glycomonolipids; and
   b. optionally isolating the glycomonolipids;
      wherein the glycomonolipids have the following formula 1:

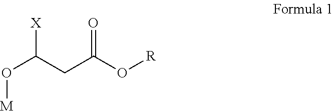

wherein X is selected from an alkyl, aryl, heteroalkyl, heteroaryl, unsaturated alkenyl, and unsaturated heteroalkenyl; and has a carbon chain length from 4 to 22;
wherein M is selected from one or two of glucose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl, and all possible stereoisomers thereof.
B. The method of paragraph A, wherein the hydrolase comprises a polypeptide sequence having at least 70% identity to SEQ ID NO: 11 or fragments thereof.
C. The method of any one of paragraph A or B, wherein the hydrolase is a carboxylic ester hydrolase selected from the group consisting of esterases (EC 3.1.1.1, EC 3.1.1.43, EC 3.1.1.84, EC 3.1.1.85, EC 3.1.1.86, EC 3.1.1.87, EC 3.1.1.112, EC 3.1.1.113, EC 3.1.1.114), lipases (EC 3.1.1.3, EC 3.1.1.23), phospholipase (EC 3.1.1.4), lysophospholipase (EC 3.1.1.5), acetylesterase (EC 3.1.1.6), depolymerase (EC 3.1.1.75, EC 3.1.1.76) cutinase (EC 3.1.1.74) and hydrolases (EC 3.1.1.22, EC 3.1.1.50, EC 3.1.1.71, EC 3.1.1.101, EC 3.1.1.102).
D. The method of any one of paragraphs A-C, wherein the hydrolase used in step a. is made by being heterologously expressed in a host cell.
E. The method of any one of Paragraphs A-D, wherein the host cell is at least one of a eukaryotic or a prokaryotic organism.
F. The method of any one of Paragraphs A-E, wherein the host cell is at least one of Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Pseudomonas putida, Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis, Tetragenococcus koreensis, Methylobacterium sp., and Methylorubrum sp.
G. The method of any one of Paragraphs A-F, wherein the glycodilipid is a rhamnodilipid and the glycomonolipid is a rhamnomonolipid.
H. The method of any one of Paragraphs A-G, wherein the rhamnodilipid is selected from Rha-C8C8, Rha-C8C10, Rha-C8C10:1, Rha-C8C12, Rha-C9C10, Rha-C10:1C8, Rha-C10:1C10, Rha-C10C8, Rha-C10C9, Rha-C10C10, Rha-C10C10:1, Rha-C10C11, Rha-C10C12, Rha-C10C12:1, Rha-C10C14, Rha-C10C14:1, Rha-C10C16, Rha-C11C10, Rha-C12:1C8, Rha-C12:1C10, Rha-C12:1C12Rha-C12:1C12:1, Rha-C12C8, Rha-C12C10, Rha-C12C12, Rha-C12C12:1, Rha-C12C14:1, Rha-C14:1C10, RhaRha-C8C8, RhaRha-C8C10, RhaRha-C8C12, RhaRha-C8C12:1, RhaRha-C10:1C10, RhaRha-C10:1C12:1, RhaRha-C10C8, RhaRha-C10C10, RhaRha-C10C10:1, RhaRha-C10C12, RhaRha-C10C12:1, RhaRha-C10C14, RhaRha-C10C14:1, RhaRha-C12:1C8, RhaRha-C12:1C10, RhaRha-C12:1C12, RhaRha-C12:1C12:1, RhaRha-C12C8, RhaRha-C12C10, RhaRha-C12C12, RhaRha-C12C12:1, RhaRha-C12C14, RhaRha-C14:1C10, RhaRha-C14C10, and combinations thereof.
I. The method of any one of Paragraphs A-H, wherein the rhamnodilipid is reacted with the hydrolase at a pH from 6 to 10; at a temperature from 25° C. to 42° C.; and under agitation in a buffered solution.
J. The method of any one of Paragraphs A-I, wherein after step a, the hydrolase is isolated from the rhamnomonolipids.
K. The method of any one of Paragraphs A-J, wherein the rhamnomonolipids do not exhibit detectable discoloration from the rhamnodilipids.
L. The method of any one of Paragraphs A-K, wherein the resulting rhamnolipid mixture produced by step a comprises at least about 75% rhamnomonolipids.
M. The method of any one of Paragraphs A-L, wherein the hydrolase comprises a polypeptide sequence having at least 90% identity to SEQ ID NO: 11 or fragments thereof.
N. The method of any one of Paragraphs A-M, wherein the hydrolase is encapsulated or immobilized.
O. A method of making rhamnomonolipids in a recombinant cell, said method comprising the following steps:
a. culturing the recombinant cell to generate rhamnomonolipids; and
b. optionally isolating the resulting rhamnomonolipids;
wherein said recombinant cell comprises:
(i) an enzyme (A) comprising the SEQ ID NO: 7;
(ii) an enzyme (B) comprising the SEQ ID NO: 8; and
(iii) an ester hydrolase comprising SEQ ID NO: 11.
P. The method according to Paragraph O, wherein enzyme (A) catalyzes the conversion of 3-OH fatty acid to 3-(Hydroxyalkanoyloxy)alkanoic acid and enzyme (B) catalyzes the addition of a single rhamnose unit to 3-(Hydroxyalkanoyloxy)alkanoic acid.
Q. The method of any one of Paragraphs O or P, wherein the recombinant cell is at least one of Pseudomonas putida, Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis Tetragenococcus koreensis, Methylobacterium sp., and Methylorubrum sp.
R. The method of any one of Paragraphs O-Q, wherein the ester hydrolase comprises a polypeptide sequence having at least 70% identity to SEQ ID NO: 11 or fragments thereof.
S. The method of any one of Paragraphs O—R, wherein the recombinant cell additionally comprises an enzyme (C) comprising of SEQ ID NO: 9.
T. The method of any one of Paragraphs O—S, wherein the making of rhamnolipids is by culturing the recombinant cell at a pH from 6 to 10; at a temperature from 25° C. to 42° C.; and under agitation in a buffered solution with a carbon feedstock.
U. The method of any one of Paragraphs O-T, where the feedstock is selected from fatty acid distillate, used soybean oil, soybean oil soapstock, orange peels, distillery waste, wheat straw, sweet water, sugarcane begasse, cellulosic waste streams, and combinations thereof.
V. The method of any one of Paragraphs O—U, wherein the recombinant cell further comprises a methyltransferase having a sequence having at least 70% identity to SEQ ID NO: or fragments thereof.
W. A recombinant cell comprising the following:
(i) an enzyme (A) comprising the SEQ ID NO: 7;
(ii) an enzyme (B) comprising the SEQ ID NO: 8; and
(iii) an ester hydrolase comprising SEQ ID NO: 11.
×. The recombinant cell of Paragraph W, wherein enzyme (A) catalyzes the conversion of 3-OH fatty acid to 3-(Hydroxyalkanoyloxy)alkanoic acid and enzyme (B) catalyzes the addition of a single rhamnose unit to 3-(Hydroxyalkanoyloxy)alkanoic acid.
Y. The recombinant cell of any one of Paragraph W or X, wherein the recombinant cell is at least one of Pseudomonas putida, Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis Tetragenococcus koreensis, Methylobacterium sp., and Methylorubrum sp.
Z. The recombinant cell of any one of Paragraphs W—Y, wherein the ester hydrolase comprises a polypeptide sequence having at least 70% identity to SEQ ID NO: 11 or fragments thereof.
AA. The recombinant cell of any one of Paragraphs W—Z, wherein the recombinant cell additionally comprises an enzyme (C) comprising of SEQ ID NO: 9.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

What is claimed is:

1. A method of making glycomonolipids; said method comprising the following steps:
a. reacting glycodilipids with a hydrolase to form glycomonolipids; and
b. optionally isolating the glycomonolipids;
wherein the glycomonolipids have the following formula 1: 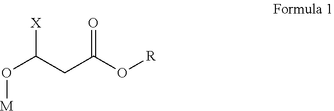
wherein X is selected from an alkyl, aryl, heteroalkyl, heteroaryl, unsaturated alkenyl, and unsaturated heteroalkenyl; and has a carbon chain length from 4 to 22;
wherein M is selected from one or two of glucose, fructose, galactose, ribose, maltose, xylose, rhamnose, sophorose, mannose, arabinose, fucose, and combinations and stereoisomers thereof; and wherein R is selected from —H, an alkyl selected from a straight-chained, branched or cyclic alkyl; a heteroalkyl; aryl; heteroaryl; hetero arylalkyl; arylalkyl; tauryl, and all possible stereoisomers thereof; and all combinations thereof.

2. The method of claim 1, wherein the hydrolase comprises a polypeptide sequence having at least 70% identity to SEQ ID NO: 11 or fragments thereof.

3. The method of claim 1, wherein the hydrolase is a carboxylic ester hydrolase selected from the group consisting of esterases (EC 3.1.1.1, EC 3.1.1.43, EC 3.1.1.84, EC 3.1.1.85, EC 3.1.1.86, EC 3.1.1.87, EC 3.1.1.112, EC 3.1.1.113, EC 3.1.1.114), lipases (EC 3.1.1.3, EC 3.1.1.23), phospholipase (EC 3.1.1.4), lysophospholipase (EC 3.1.1.5), acetylesterase (EC 3.1.1.6), depolymerase (EC 3.1.1.75, EC 3.1.1.76) cutinase (EC 3.1.1.74) and hydrolases (EC 3.1.1.22, EC 3.1.1.50, EC 3.1.1.71, EC 3.1.1.101, EC 3.1.1.102).

4. The method of claim 1, wherein the hydrolase used in step a. is made by being heterologously expressed in a host cell.

5. The method of claim 4, wherein the host cell is at least one of a eukaryotic or a prokaryotic organism.

6. The method of claim 4, wherein the host cell is at least one of Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Pseudomonas putida, Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis, Tetragenococcus koreensis, Methylobacterium sp., and Methylorubrum sp.

7. The method of claim 1, wherein the glycodilipid is a rhamnodilipid and the glycomonolipid is a rhamnomonolipid.

8. The method of claim 7, wherein the rhamnodilipid is selected from Rha-C8C8, Rha-C8C10, Rha-C8C10:1, Rha-C8C12, Rha-C9C10, Rha-C10:1C8, Rha-C10:1C10, Rha-C10C8, Rha-C10C9, Rha-C10C10, Rha-C10C10:1, Rha-C10C11, Rha-C10C12, Rha-C10C12:1, Rha-C10C14, Rha-C10C14:1, Rha-C10C16, Rha-C11C10, Rha-C12:1C8, Rha-C12:1C10, Rha-C12:1C12Rha-C12:1C12:1, Rha-C12C8, Rha-C12C10, Rha-C12C12, Rha-C12C12:1, Rha-C12C14:1, Rha-C14:1C10, RhaRha-C8C8, RhaRha-C8C10, RhaRha-C8C12, RhaRha-C8C12:1, RhaRha-C10:1C10, RhaRha-C10:1C12:1, RhaRha-C10C8, RhaRha-C10C10, RhaRha-C10C10:1, RhaRha-C10C12, RhaRha-C10C12:1, RhaRha-C10C14, RhaRha-C10C14:1, RhaRha-C12:1C8, RhaRha-C12:1C10, RhaRha-C12:1C12, RhaRha-C12:1C12:1, RhaRha-C12C8, RhaRha-C12C10, RhaRha-C12C12, RhaRha-C12C12:1, RhaRha-C12C14, RhaRha-C14:1C10, RhaRha-C14C10, and combinations thereof.

9. The method of claim 7, wherein the rhamnodilipid is reacted with the hydrolase at a pH from 6 to 10; at a temperature from 25° C. to 42° C.; and under agitation in a buffered solution.

10. The method of claim 7, wherein after step a, the hydrolase is isolated from the rhamnomonolipids and optionally reused in subsequent hydrolyses.

11. The method of claim 7, wherein the rhamnomonolipids do not exhibit detectable discoloration from the rhamnodilipids.

12. The method of claim 8, wherein the resulting rhamnolipid mixture produced by step a comprises at least about 75% rhamnomonolipids.

13. The method of claim 1, wherein the hydrolase comprises a polypeptide sequence having at least 90% identity to SEQ ID NO: 11 or fragments thereof.

14. The method of claim 1, wherein the hydrolase is encapsulated or immobilized.

15. A method of making rhamnomonolipids in a recombinant cell, said method comprising the following steps:
a. culturing the recombinant cell to generate rhamnomonolipids; and
b. optionally isolating the resulting rhamnomonolipids;
wherein said recombinant cell comprises:
(i) an enzyme (A) comprising SEQ ID NO: 7;
(ii) an enzyme (B) comprising SEQ ID NO: 8; and
(iii) an ester hydrolase comprising SEQ ID NO: 11.

16. The method according to claim 15, wherein enzyme (A) catalyzes the conversion of 3-OH fatty acid to 3-(Hydroxyalkanoyloxy)alkanoic acid and enzyme (B) catalyzes the addition of a single rhamnose unit to 3-(Hydroxyalkanoyloxy)alkanoic acid.

17. The method of claim 15, wherein the recombinant cell is at least one of Pseudomonas putida, Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis Tetragenococcus koreensis, Methylobacterium sp., and Methylorubrum sp.

18. The method of claim 15, wherein the ester hydrolase comprises a polypeptide sequence having at least 70% identity to SEQ ID NO: 11 or fragments thereof.

19. The method of claim 15, wherein the recombinant cell additionally comprises an enzyme (C) comprising SEQ ID NO: 9.

20. The method of claim 15, wherein the making of rhamnolipids is by culturing the recombinant cell at a pH from 6 to 10; at a temperature from 25° C. to 42° C.; and under agitation in a buffered solution with a carbon feedstock.

21. The method of claim 20, where the feedstock is selected from fatty acid distillate, used soybean oil, soybean oil soapstock, orange peels, distillery waste, wheat straw, sweet water, sugarcane begasse, cellulosic waste stream and combinations thereof.

22. The method of claim 15, wherein the recombinant cell further comprises a methyltransferase having a sequence having at least 70% identity to SEQ ID NO: 10 or fragments thereof.

23. A recombinant cell comprising the following:
(i) an enzyme (A) comprising the SEQ ID NO: 7;
(ii) an enzyme (B) comprising the SEQ ID NO: 8; and
(iii) an ester hydrolase comprising SEQ ID NO: 11.

24. The recombinant cell of claim 23, wherein enzyme (A) catalyzes the conversion of 3-OH fatty acid to 3-(Hydroxyalkanoyloxy)alkanoic acid and enzyme (B) catalyzes the addition of a single rhamnose unit to 3-(Hydroxyalkanoyloxy)alkanoic acid.

25. The recombinant cell of claim 23, wherein the recombinant cell is at least one of Pseudomonas putida, Escherichia coli, Bacillus subtilis, Streptomyces coelicolor, Pseudomonas aeruginosa, Pichia pastoris, Saccharomyces cerevisiae, Kluyveromyces lactis, Aspergillus niger, Trichoderma reesei, Corynebacterium sp., Burkholderia thailandensis, Parabulkholderia sp., Acinetobacter sp., Alcanivorax sp., Antarctobacter sp., Bacillus sp., Burkholderia sp., Candida apicola, Cellulomonas cellulans, Cupriavidus necator, Enterobacter sp., Halomonas sp., Lactobacilli sp., Marinobacter sp., Myxococcus sp., Nocardioides sp., Pseudoalteromonas sp., Pseudomonas sp., Pseudoxanthomonas sp., Renibacterium salmoninarum, Rhodoccus sp., Rhodotorula bogoriensis Tetragenococcus koreensis, Methylobacterium sp., and Methylorubrum sp.

26. The recombinant cell of claim 23, wherein the ester hydrolase comprises a polypeptide sequence having at least 70% identity to SEQ ID NO: 11 or fragments thereof.

27. The recombinant cell of claim 23, wherein the recombinant cell additionally comprises an enzyme (C) comprising SEQ ID NO: 9.
